(19) United States
(12) Patent Application Publication
(10) Pub. No.: US 2026/0354678 A1
(43) Pub. Date: Nov. 12, 2026

(54) HUMAN POWERED SOFT ROBOTICS FOR NEUROMUSCULAR DISORDERS

(71) Applicant: VIRGINIA TECH INTELLECTUAL PROPERTIES, INC., Blacksburg, VA (US)

(72) Inventors: Christopher ARENA, Blacksburg, VA (US); William TRADER, Advance, NC (US)

(21) Appl. No.: 19/490,635

(22) PCT Filed: Jun. 7, 2024
(86) PCT No.: PCT/US2024/032962

§ 371 Date: Dec. 5, 2025

Related U.S. Application Data
(60) Provisional application No. 63/471,893, filed on Jun. 8, 2023.

(51) Int. Cl. A61B 3/00; A61B 5/00; A61B 5/103; A61F 5/14

(52) U.S. Cl. CPC A61B 3/00; A61B 5/1038; A61B 5/6807; A61F 5/14; A61B 2562/168; A61H 2201/0103; A61H 2201/1238; A61H 2230/625

(57) ABSTRACT

An apparatus may include at least one sensor bladder for positioning in relation to the foot of a user. An apparatus may also include at least one stimulation bladder corresponding to the respective at least one sensor bladder, the at least one stimulation bladder positioned on the body of the user remotely from the foot. An apparatus may include at least one conduit fluidly coupling the respective at least one sensor bladder with the respective at least one stimulation bladder, where the at least one sensor bladder is configured to compress responsive to movement of the foot of the user and drive fluid from the at least one sensor bladder to the respective at least one stimulation bladder via the respective at least one conduit.

### CROSS REFERENCE TO RELATED APPLICATIONS

This application claims the benefit of and priority to U.S. Provisional Application Ser. No. 63/471,893, filed Jun. 8, 2023, titled “HUMAN POWERED SOFT ROBOTICS FOR NEUROMUSCULAR DISORDERS.” the entire contents of which are hereby incorporated herein by reference.

### TECHNICAL FIELD

This disclosure relates to soft robotics, and in particular, to use of soft robotics to address peripheral neuropathy.

### DESCRIPTION OF THE RELATED TECHNOLOGY

Peripheral neuropathy can be caused by damage to peripheral nerves. In most instances, peripheral neuropathy can begin distally and over time spread up the limbs. Peripheral neuropathy in the lower extremities can be associated with numbness of the feet, which can cause reduced gait speed, reduced balance, and increased fall risk. Electronically controlled robotics can be employed to address some of these ailments.

### SUMMARY

In some aspects, the techniques described herein relate to an apparatus, including: at least one sensor bladder for positioning in relation to the foot of a user: at least one stimulation bladder corresponding to the respective at least one sensor bladder, the at least one stimulation bladder positioned on the body of the user remotely from the foot; and at least one conduit fluidly coupling the respective at least one sensor bladder with the respective at least one stimulation bladder, wherein the at least one sensor bladder is configured to compress responsive to movement of the foot of the user and drive fluid from the at least one sensor bladder to the respective at least one stimulation bladder via the respective at least one conduit.

In some aspects, the techniques described herein relate to an apparatus, wherein the at least one sensor bladder includes a forefoot sensor bladder for positioning under the forefoot of the user and a hindfoot sensor bladder for positioning under the hindfoot of the user.

In some aspects, the techniques described herein relate to an apparatus, wherein the at least one sensor bladder includes a first forefoot sensor bladder for positioning under a first portion of the forefoot of the user, a second forefoot sensor bladder for positioning under a second portion of the forefoot, and a hindfoot sensor bladder for positioning under the hindfoot of the user.

In some aspects, the techniques described herein relate to an apparatus, wherein the first portion of the forefoot includes the bottom surface of the toes of the foot and the second portion of the forefoot includes a bottom surface of the foot between the toes and the heel, and the hindfoot of the user includes the heel of the foot.

In some aspects, the techniques described herein relate to an apparatus, wherein the first portion of the forefoot includes a lateral forefoot portion and the second portion of the forefoot includes a medial forefoot portion.

In some aspects, the techniques described herein relate to an apparatus, further including: a shoe insert having a foot facing surface, the at least one sensor bladder attached to the foot facing surface, the shoe insert including: a side surface defining at least one conduit port for coupling with the respective one of the at least one conduit.

In some aspects, the techniques described herein relate to an apparatus, further including: a shoe insert having a foot facing surface and a opposite shoe facing surface, the at least one sensor bladder attached to the shoe facing surface, the shoe insert including: a side surface defining at least one conduit port for coupling with the respective one of the at least one conduit.

In some aspects, the techniques described herein relate to an apparatus, further including: a shoe for receiving the shoe insert, the shoe including a sole having an outer sidewall that defines at least one opening to allow the at least one conduit to pass through the sole and couple with the at least one conduit port.

In some aspects, the techniques described herein relate to an apparatus, further including: a lower extremity garment having an inner surface and an outer surface, wherein the at least one sensor bladder is attached to the outer surface or the inner surface of the lower extremity garment.

In some aspects, the techniques described herein relate to an apparatus, further including: a lower extremity garment having an inner surface and an outer surface, wherein the at least one sensor bladder is attached to the outer surface or the inner surface of the lower extremity garment.

In some aspects, the techniques described herein relate to an apparatus, wherein the lower extremity garment comprises a sock, a compression sleeve, or a brace; and/or the lower extremity garment is machine-washable or waterproof.

In some aspects, the techniques described herein relate to an apparatus, wherein the lower extremity garment includes an upper portion that extends beyond the ankle of the foot and wherein the at least one stimulation bladder is attached to the upper portion of the lower extremity garment.

In some aspects, the techniques described herein relate to an apparatus, wherein the at least one sensor bladder includes a first membrane and a second membrane and wherein portions of the inner surfaces of the first membrane and the second membrane are attached to each other.

In some aspects, the techniques described herein relate to an apparatus, further comprising one or more walls that extend between a first inner surface and an opposing second inner surface of the at least one sensor bladder, and wherein each bladder of the at least one sensor bladder has a contiguous volume.

In some aspects, the techniques described herein relate to an apparatus, wherein the at least one stimulation bladder comprises one or more walls that extend inwardly from a perimeter of the at least one stimulation bladder.

In some aspects, the techniques described herein relate to an apparatus, wherein the at least one sensor bladder includes an expander positioned between two inner surfaces, wherein the expander is configured to compress responsive to compressive force from the foot of the user and to expand and draw fluid back into the at least one sensor bladder responsive to reduction in compressive force from the foot.

In some aspects, the techniques described herein relate to an apparatus, further including a wearable leg-wrap with elastic lining, wherein the at least one stimulation bladder is positioned between the wearable leg-wrap and the elastic lining.

In some aspects, the techniques described herein relate to an apparatus, wherein the at least one stimulation bladder incudes a ring-shaped bladder wrapped circumferentially around the leg of the user.

In some aspects, the techniques described herein relate to an apparatus, wherein the at least one stimulation bladder includes two or more ring-shaped bladders wrapped circumferentially around the leg of a user.

In some aspects, the techniques described herein relate to an apparatus 17 and 18 wherein the at least one stimulation bladder includes McKibben air muscles.

In some aspects, the techniques described herein relate to an apparatus 17 and 18, wherein the at least one stimulation bladder includes pneumatic artificial muscles.

In some aspects, the techniques described herein relate to an apparatus, wherein the at least one stimulation bladder includes two or more stimulation bladders, each having a width that is less than a half the circumference of a portion of the leg where the respective one of the two or more stimulation bladders is disposed.

In some aspects, the techniques described herein relate to an apparatus, wherein the at least one stimulation bladder includes a plurality of stimulation bladders and wherein the plurality of stimulation bladders are positioned on the leg of the user at approximately the same distance from the base of the foot.

In some aspects, the techniques described herein relate to an apparatus, wherein the at least one stimulation bladder includes a plurality of stimulation bladders and wherein the plurality of stimulation bladders are positioned on the leg of the user at different distances from the base of the foot.

In some aspects, the techniques described herein relate to an apparatus, wherein the at least one stimulation bladder includes a plurality of stimulation bladders and wherein at least two of the plurality of stimulation bladders are positioned on opposite sides of the leg of the user.

In some aspects, the techniques described herein relate to an apparatus, wherein the at least one stimulation bladder includes a wall positioned in front of a fluid port of the at least one stimulation bladder.

In some aspects, the techniques described herein relate to an apparatus, wherein the fluid includes air.

In some aspects, the techniques described herein relate to an apparatus, wherein the fluid incudes a liquid.

In some aspects, the techniques described herein relate to an apparatus, further including at least one valve coupled with the respective one of at least one conduit to allow for fluid to be removed or entered.

In some aspects, the techniques described herein relate to an apparatus, wherein the at least one sensor bladder is formed of material including thermoplastic polyurethane film or thermoset plastics.

In some aspects, the techniques described herein relate to an apparatus, wherein the at least one conduit is formed of material including perfluoroalkoxy alkanes (PFA) or thermoset plastics.

In some aspects, the techniques described herein relate to an apparatus, wherein the at least one sensor bladder, the at least one stimulation bladder, and the at least one conduit form an integrated unit, the apparatus further including a valve attached to an opening into the integrated unit.

In some aspects, the techniques described herein relate to an apparatus, wherein the at least one sensor bladder includes a tubing portion having a first end and a second end, the first end being broader than the second end, the first end opening into the interior of the at least one sensor bladder and the second end coupling with the at least one conduit.

In some aspects, the techniques described herein relate to an apparatus, further including at least one wall positioned within the tubing portion extending between the first end and the second end.

In some aspects, the techniques described herein relate to an apparatus, wherein the at least one stimulation bladder is positioned anywhere at the body of the user.

In some aspects, the techniques described herein relate to an apparatus, wherein the at least one sensor bladder and the at least one stimulation bladder are shaped differently.

In some aspects, the techniques described herein relate to an apparatus, wherein each of the one or more walls form a hollow slot.

In some aspects, the techniques described herein relate to an apparatus, wherein the hollow slot is substantially rectangular in shape.

In some aspects, the techniques described herein relate to an apparatus, further comprising a wearable leg-wrap with elastic lining, wherein the at least one stimulation bladder or the at least one sensor bladder is sandwiched by the wearable leg-wrap against a leg of the user.

In some aspects, the techniques described herein relate to an apparatus, including: a first sensor bladder positioned behind a knee of a user, the first sensor bladder configured to compress when the user is about to take a step forward; a actuation bladder extending between the ankle and the top of a foot of the user, the actuation bladder configured to contract in length in response to pressurization, and a conduit extending between the first sensor bladder and the actuation bladder.

In some aspects, the techniques described herein relate to an apparatus, wherein the first sensor bladder is positioned behind the knee of a first leg of the user, and wherein the actuation bladder is positioned on the foot of a second leg of the user.

In some aspects, the techniques described herein relate to an apparatus, wherein the first sensor bladder is positioned under the foot of a first leg of the user, and wherein the actuation bladder is positioned on the foot of a second leg of the user.

### BRIEF DESCRIPTION OF THE DRAWINGS

FIG. 1 shows a schematic of an example soft robotics system that includes at least one sensor bladder, at least one stimulation bladder, and at least one conduit.

FIG. 2 shows activations of sensor and stimulation bladders depicted in FIG. 1 for various stances of the user.

FIGS. 3A and 3B show additional example configurations of the at least one sensor bladder.

FIG. 4 shows an example of at least one stimulation bladders as ring-shaped bladders.

FIGS. 5A-5C show various positions for one or more stimulation bladders.

FIG. 6 shows a portion of an example soft robotics system where at least one conduit that fluidly couples at least one sensor bladder with at least one stimulation bladder is coupled with a shoe of the user.

FIG. 7 shows an example configuration of at least one sensor bladder.

FIG. 8 shows a photograph depicting top views of manufactured sensor bladders.

FIG. 9 shows an example at least one sensor bladder with an expander.

FIGS. 10A and 10B show another example configuration of at least one sensor bladder.

FIG. 11 shows an example structure of the one or more bladders discussed herein.

FIG. 12 shows a photograph of an example set of sensor bladders.

FIG. 13 shown an example stimulation bladder housing attached to a user's leg.

FIG. 14 shows a portion of a cross-sectional view of the example stimulation bladder housing discussed above in relation to FIG. 13.

FIG. 15 shows a cross-sectional view of the example stimulation bladder housing discussed above in relation to FIG. 13.

FIG. 16 shows atop view of an example stimulation bladder.

FIG. 17 shows graphical representation of forces measured at the various sensor bladders of an example soft robotics system.

FIGS. 18-20 show additional graphical representations of forces measured at various sensor and stimulation bladders.

FIG. 21 shows a schematic of an example bladder system.

FIG. 22 shows schematics of another example soft robotics system for addressing drop-foot.

FIG. 23 shows another example bladder with a tubing portion.

Like reference numbers and designations in the various drawings indicate like elements.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about.” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.10% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about.” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Prior to describing the various aspects of the present disclosure, the following definitions are provided and should be used unless otherwise indicated. Additional terms may be defined elsewhere in the present disclosure.

As used herein. “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “a proton beam degrader,” “a degrader foil,” or “a conduit,” includes, but is not limited to, two or more such proton beam degraders, degrader foils, or conduits, and the like.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere).

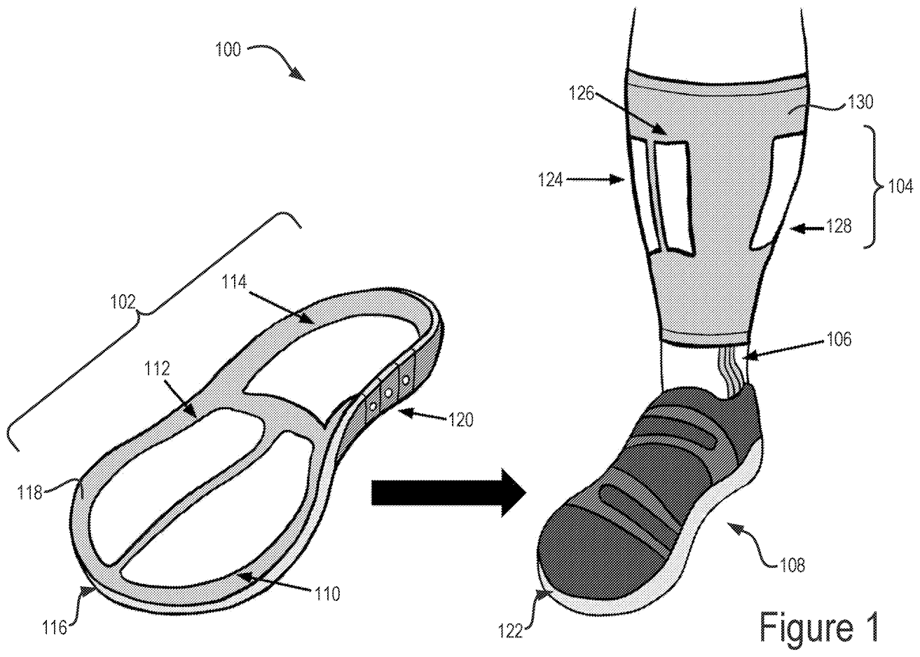

FIG. 1 shows a schematic of an example soft robotics system 100 that includes at least one sensor bladder 102, at least one stimulation bladder 104, and at least one conduit 106. The at least one sensor bladder 102 can be positioned in relation to the foot 108 of a user such as a patient or any person that is capable of using or implementing the soft robotics system 100. For example, the at least one sensor bladder 102 can be positioned below the foot of the user. The at least one stimulation bladder 104 can be positioned on the body remotely from the foot 108. For example, the at least one stimulation bladder 104 can be positioned on the leg above the ankle. The at least one conduit 106 fluidly couples the at least one sensor bladder 102 with the at least one stimulation bladder 104. In particular, each of the at least one conduit 106 fluidly couples one of the at least one sensor bladder 102 with a respective one of the at least one stimulation bladder 104.

In the example shown in FIG. 1, the at least one sensor bladder 102 includes three sensor bladders: a medial forefoot sensor bladder 110 (also referred to as “a first forefoot sensor bladder”), a lateral forefoot sensor bladder 112 (also referred to as “a second forefoot sensor bladder”) and a hindfoot sensor bladder 114. The number of sensor bladders can be different from the three shown in FIG. 1. For example, the number of sensor bladders can range from one sensor bladder to tens of sensor bladders. The medial forefoot sensor bladder 110 is positioned under a first portion of the forefoot on the median side of a para-sagittal plane that includes a longitudinal axis that runs between the forefoot and the heel. The lateral forefoot sensor bladder 112 is positioned under a second portion of the forefoot on the lateral side of the para-sagittal plane. The hindfoot sensor bladder 114 is positioned posterior to both the medial forefoot sensor bladder 110 and the lateral forefoot sensor bladder 112.

The position, shape, and size of each of the at least one sensor bladder 102 shown in FIG. 1 are only examples and can vary with the particular implementation. In some instances, for example, the position, shape, and size of each the at least one sensor bladder 102 can be based on the topology of the neuropathy under the foot of the user. In some such instances, the at least one sensor bladder 102 can be positioned under the portions of the foot of the user where the user has lost or has compromised sensory perception. Further, the shape and the size of each one of the at least one sensor bladder 102 can be selected to cover the desired portions of the plantar of the foot. In the example shown in FIG. 1, the shape of the bladders is selected to conform to the shape of the sole of the foot of the user. The size of the bladders is selected to cover substantially the entire surface area of the sole. However, in other examples, the size and shape of the bladders can be different than that shown in FIG. 1. For example, the bladders may not conform to the shape of the sole, and instead include circular, elliptical, rectangular, or polygonal in shape, and be positioned within the desired area of the foot. Additional examples of positions, shapes, and sizes of the bladders is discussed further below.

Referring again to FIG. 1, the medial forefoot sensor bladder 110, the lateral forefoot sensor bladder 112, and the hindfoot sensor bladder 114 are adhered to a shoe insert 116 or orthotic, which can be positioned inside a shoe of the user. The bladders can be positioned within the periphery of the shoe insert 116 and can be adhered to a foot facing surface 118 of the shoe insert 116 by using glue or other fastening means. The shoe insert 116 can include conduit ports 120 with which the at least one conduit 106 can be coupled. In the example shown in FIG. 1, the at least one sensor bladder 102 includes three bladders, and the shoe insert 116 includes three conduit ports 120 corresponding to the three bladders. In some examples, a bladder can be coupled with a single port (of the conduit ports 120). In some other examples, a bladder can be coupled with two or more ports. In the example shown in FIG. 1, the each of the three bladders are coupled with one port of the conduit ports 120. The shoe insert 116 can include channels that couple the bladders with the conduit ports 120. For example, the shoe insert can include three channels that fluidly couple the three bladders with the three conduit ports 120 respectively. The channels in the shoe insert 116 can be embedded within the shoe insert 116 such as, for example, below the foot facing surface 118 or can be routed along the side of the shoe insert 116. Having the channels embedded can reduce the risk of discomfort that may be caused by channels that run along the side of the shoe insert 116 and which may come in contact with the foot of the user. The shoe insert 116 may be a removeable shoe insert of a shoe 122 or can be a permanent shoe insert of the shoe 122. In some instances, the bladders may be removably adhered to the shoe insert 116 to allow for replacement and adjustment of position of the bladders. In some examples, the bladders can be adhered to a lower extremity garment such as sock instead of to the shoe insert 116 or to the inside of the shoe 122 of the user. Other examples of the lower extremity garment can include a compression sleeve, a brace, or any other type of garment that may be wearable or attachable on the user's leg. The benefit of the lower extremity garment is that it can be machine washable and/or waterproof, facilitating reusability and ease of use for the bladders described herein. As discussed herein, the fluid can include gaseous and/or liquid matter such as, for example, air, nitrogen, water, oil, etc. In some instances, the fluid can also include solid suspended particles or gels.

In the example shown in FIG. 1, the at least one stimulation bladder 104 can include three stimulation bladders corresponding to the three sensor bladders. In particular, the at least one stimulation bladder 104 can include three stimulation bladders: a lateral forefoot stimulation bladder 124, a medial forefoot stimulation bladder 126, and a hindfoot stimulation bladder 128. The lateral forefoot stimulation bladder 124 corresponds to and can be in fluid communication with the lateral forefoot sensor bladder 112, the medial forefoot stimulation bladder 126 corresponds to and can be in fluid communication with the medial forefoot sensor bladder 110, and the hindfoot stimulation bladder 128 corresponds to and can be in fluid communication with the hindfoot sensor bladder 114. The at least one conduit 106 includes three conduits, where a first conduit has a first end fluidly coupled with the lateral forefoot sensor bladder 112 and a second end fluidly coupled with the lateral forefoot stimulation bladder 124, where a second conduit has a first end fluidly coupled with medial forefoot sensor bladder 110 and a second end fluidly coupled with the medial forefoot stimulation bladder 126, and where a third conduit has a first end fluidly coupled with the hindfoot sensor bladder 114 and a second end fluidly coupled with the hindfoot stimulation bladder 128. The conduits allow fluid flow between a sensor bladder and the respective stimulation bladder.

The at least one stimulation bladder 104 can be positioned remotely from the foot of the user. In the example shown in FIG. 1, the at least one stimulation bladder 104 is positioned above the ankle and below the knee of the user. However, in some other examples, the at least one stimulation bladder 104 can be positioned at other locations on the user's body such as, for example, above the knee, the arms, the torso, or any other location where the user has full or partial sense perception. Where the at least one stimulation bladder 104 includes a plurality of stimulation bladders, one or more of the stimulation bladders can be distributed over multiple parts of the user's body. The three stimulation bladders are positioned at approximately the same height from the bottom surface of the foot. Positioning the stimulation bladders are the same height can help in equalizing the time difference between the stimulation felt at different stimulation bladders when the corresponding sensor bladders are simultaneously activated. However, this is only an example, and the stimulation bladders can be positioned at different heights from the bottom surface of the foot. In some instances, the positions of the stimulation bladders can be dictated by the ability of the simulation bladders to create an effective sense perception on the skin of the user. Each stimulation bladder may be positioned greater than a threshold distance (e.g., 0.3 inches-0.8 inches, or about 0.5 inches) from an adjacent stimulation bladder. The threshold distance ensures that the user can separately discern or sense the stimulation received from adjacent stimulation bladders. In some instances, the stimulation bladders can be placed as far as possible from each other. For example, the three stimulation bladders shown in FIG. 1 may be spaced apart by about ⅓^{rd} of the circumference of the portion of the leg around which the three stimulation bladders are positioned.

The shape and the size of the at least one stimulation bladder 104 can be rectangular, as depicted in the example shown in FIG. 1. In some examples, other shapes can also be utilized such as, for example, square, circular, elliptical, polygonal, etc. in some instances, each one of the at least one stimulation bladder 104 can have the same shape and size. In some examples, two or more of the at least one stimulation bladder 104 can have different shapes or sizes. In some instances, the shape and the size of the at least one stimulation bladder 104 can be based on a number of factors such as, for example, the position of the stimulation bladder, the sensitivity of the user at the position of the stimulation bladder, the number of stimulation bladders, and the volume of the stimulation bladder in relation to the volume of the corresponding sensor bladder. Another shape can include a ring-shaped stimulation bladder that can wrap around at least a portion of the leg where the bladder is positioned. In the example shown in FIG. 1, the at least one stimulation bladder 104 can be adhered to a wearable leg-wrap 130, which can be worn by the user and positioned on the leg of the user at the desired location. The wearable leg-wrap 130 can support the expansion and contraction of the at least one stimulation bladder 104 as well as allow the at least one conduit 106 to couple with the at least one stimulation bladder 104. In some instances, the at least one stimulation bladder 104 can be removably adhered to the skin of the user, alleviating the need for a leg-wrap, which in some cases, such as diabetic users, may not be recommended.

The shoe insert 116 shown in FIG. 1 can also include a shoe facing surface that is on the other side of the shoe insert, opposite from the foot facing surface 118. In some instances, the at least one sensor bladder 102 can be attached to the shoe facing surface of the shoe insert 116. In some instances, where the at least one sensor bladder 102 includes more than one bladder, at least one bladder can be positioned on each of the foot facing surface 118 and the shoe facing surface. When the shoe insert 116 is inserted into the shoe, a bladder positioned on the shoe facing surface of the shoe insert 116 would be positioned between the shoe insert 116 and the sole of the shoe. For example, the medial forefoot sensor bladder 110 and the lateral forefoot sensor bladder 112 can be positioned, as shown in FIG. 1, on the foot facing surface 118 of the shoe insert 116, while the hindfoot sensor bladder 114 can be positioned on the shoe facing surface of the shoe insert 116.

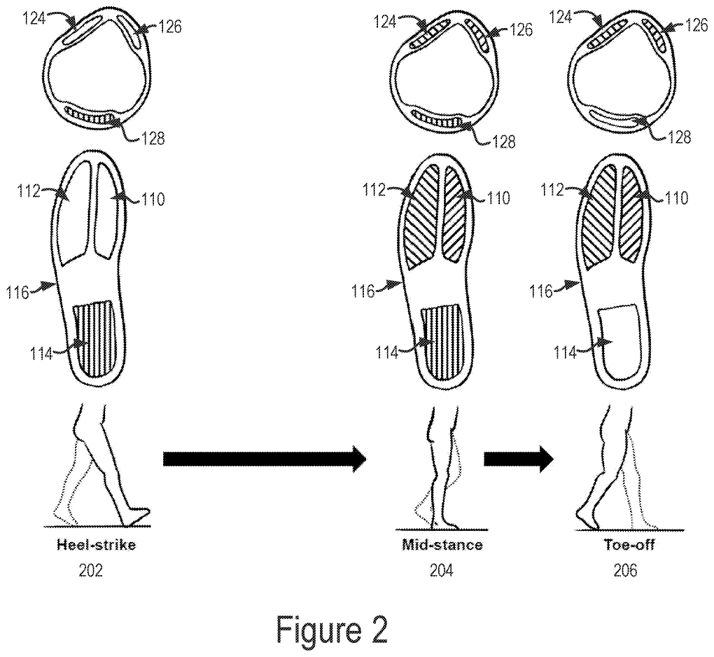

FIG. 2 shows activations of sensor and stimulation bladders depicted in FIG. 1 for various stances of the user. In particular, FIG. 2 shows the activations at three different positions in the stride of the user. A first position 202 corresponds to a heel-strike, a second position 204 corresponds to a mid-stance, and a third position 206 corresponds to a toe-off. While FIG. 2 shows positions in relation to the right leg of the user, similar operation may apply for the left leg as well. For each position, FIG. 2 shows a top view of the at least one sensor bladder 102 and a cross-sectional view of the at least one stimulation bladder 104 positioned around the leg of the user. In the first position 202, the leg of the user is in a heel-strike position, and presses on the hindfoot sensor bladder 114, but does not substantially press on the lateral forefoot sensor bladder 112 and the medial forefoot sensor bladder 110. The compression of the hindfoot sensor bladder 114 by the hindfoot of the user can cause fluid from the hindfoot sensor bladder 114 to pushed into or transferred into the hindfoot stimulation bladder 128 via the connecting conduit. The transfer of the fluid into the hindfoot stimulation bladder 128 can cause the hindfoot stimulation bladder 128 to expand and exert a force on the portion of the leg of the user positioned under the hindfoot stimulation bladder 128. This force can be felt by the user and can serve as an indicator that the user is pressing down on the ground with her/his heel.

In the second position 204, the leg of the user is in the mid-stance position, in which the entire sole of the foot is pressed down on the ground. As a result, the foot presses on each of the hindfoot sensor bladder 114, the lateral forefoot sensor bladder 112 and the medial forefoot sensor bladder 110. The compression of these bladders can cause fluid in these bladders to be forced into their corresponding stimulation bladders. For example, the pressing of the lateral forefoot sensor bladder 112 can cause fluid in the lateral forefoot sensor bladder 112 to flow into the lateral forefoot stimulation bladder 124 and the pressing of the medial forefoot sensor bladder 110 can cause the fluid in the medial forefoot sensor bladder 110 to flow into the medial forefoot stimulation bladder 126. The fluid flow from the sensor bladders into the respective stimulation bladders can cause the stimulation bladders to expand and exert force on the leg of the user. These forces can serve as indicators that the heel, and the forefoot of the user is pressing down on the ground.

In the third position 206, the leg of the user is in the toe-off position, in which the user's forefoot is pressed down on the ground, while the hindfoot does not make contact with the ground. As a result, the foot presses on each of the medial forefoot sensor bladder 110 and the lateral forefoot sensor bladder 112. Because the hindfoot sensor bladder 114 is not compressed in the third position 206, the fluid which was previously pushed into the hindfoot stimulation bladder 128 returns into the hindfoot sensor bladder 114. As a result, the hindfoot stimulation bladder 128 contacts and ceases to apply any appreciable force onto the leg of the user. The lack of pressure under the hindfoot stimulation bladder 128 serves as an indicator to the user that the hindfoot is no longer in contact with the ground. In addition, the maintenance of the pressure by the lateral forefoot stimulation bladder 124 and the medial forefoot stimulation bladder 126 provide an indication to the user that the forefoot is still pressing down on the ground.

In transitioning form one position to another such as, for example, any two positions shown in FIG. 2, the magnitude of force that the stimulation bladders exert on the leg of the user can vary based on the extent to which the respective sensor bladder has been depressed. As an example, the user may depress the hindfoot sensor bladder 114 to a greater extent when in the second position 204 (mid-stance) than in the first position 202 (heel-strike). Therefore, more fluid would be transferred to the hindfoot stimulation bladder 128 in the second position 204 than in the first position 202. As a result, the user would feel a greater force when in the second position 204 than in the first position 202. It can be noted that the magnitude of force exerted by the hindfoot stimulation bladder 128 on the user's leg can gradually increase in magnitude as the user moves from the first position 202 to the second position 204, and gradually decrease as the user moves from the second position 204 to the third position 206 when no pressure is felt. Similarly, the user may depress the medial forefoot sensor bladder 110 and the lateral forefoot sensor bladder 112 to a greater extend when in the second position 204 and the third position 206 than in the first position 202. Therefore, more fluid would be transferred to the medial forefoot stimulation bladder 126 and the lateral forefoot stimulation bladder 124 in the second position 204 or third position 206 than in the first position 202. The magnitude of forces exerted on the lateral forefoot stimulation bladder 124 and the medial forefoot stimulation bladder 126 may increase gradually when the user transitions between the first position 202 and the second position 204, and can gradually decrease when the user transitions from the third position 206 to the first position 202.

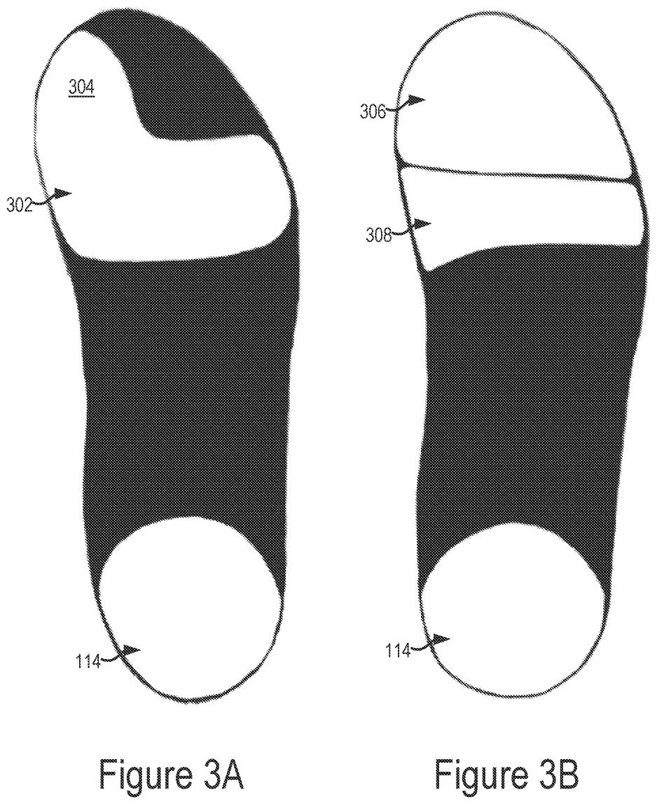

FIGS. 3A and 3B show additional example configurations of the at least one sensor bladder. In particular, FIG. 3A shows only two sensor bladders: a hindfoot sensor bladder 114 and a single forefoot sensor bladder 302. Unlike the soft robotics system 100 shown in FIG. 1 in which the one or more sensor bladders included two sensor bladders (medial forefoot sensor bladder 110 and lateral forefoot sensor bladder 112), the configuration shown in FIG. 3A include only a single forefoot sensor bladder 302. The single forefoot sensor bladder 302 can be positioned under the forefoot of the user. The single forefoot sensor bladder 302 is spaced apart from the hindfoot sensor bladder 114. The single forefoot sensor bladder 302 can be shaped such that it includes a big toe portion 304 that is positioned below the big toe of the foot and not have any portion positioned under the remainder of the toes. In some other implementations, the single forefoot sensor bladder 302 may conform to the perimeter of the forefoot of the user such that at least a portion of the single forefoot sensor bladder 302 is positioned under each toe of the user. Of course, some other implementations may shape the single forefoot sensor bladder 302 to have a portion that is positioned below a subset of the toes of the user. Shaping the sensor bladder in this manner can allow targeted sensing where, for example, only a subset of the toes of the user are neuropathic.

FIG. 3B shows three sensor bladders: a hindfoot sensor bladder 114, an anterior forefoot sensor bladder 306, and a posterior forefoot sensor bladder 308. The anterior forefoot sensor bladder 306 can be positioned closer to the toes of the foot than the posterior forefoot sensor bladder 308. The posterior forefoot sensor bladder 308 can be positioned between the hindfoot sensor bladder 114 and the anterior forefoot sensor bladder 306. As an example, the anterior forefoot sensor bladder 306 can be positioned under the toes while the posterior forefoot sensor bladder 308 can be positioned under the metatarsal. The anterior forefoot sensor bladder 306 and the posterior forefoot sensor bladder 308 can be shaped such that at least a portion of each of their perimeter conforms to the shape of the forefoot. The anterior forefoot sensor bladder 306 can be positioned abutting the posterior forefoot sensor bladder 308, or the anterior forefoot sensor bladder 306 and posterior forefoot sensor bladder 308 can be spaced apart.

Positioning of the sensor bladders can depend, in part, upon the nature of the neuropathy of the user and/or the type of sensory input desired. For example, the arrangement of the forefoot sensor bladders shown in FIG. 1, in which the medial forefoot sensor bladder 110 and the lateral forefoot sensor bladder 112 are positioned side-by-side, can provide feedback to aid user's balance. The arrangement of the forefoot sensor bladders shown in FIG. 3B, in which the anterior forefoot sensor bladder 306 and posterior forefoot sensor bladder 308 are positioned front-back in relation to the forefoot, can provide feedback for toe-off. While only a single hindfoot sensor bladder is shown in FIGS. 1, 3A, and 3B, it should be noted that more than one hindfoot sensor bladder can also be employed.

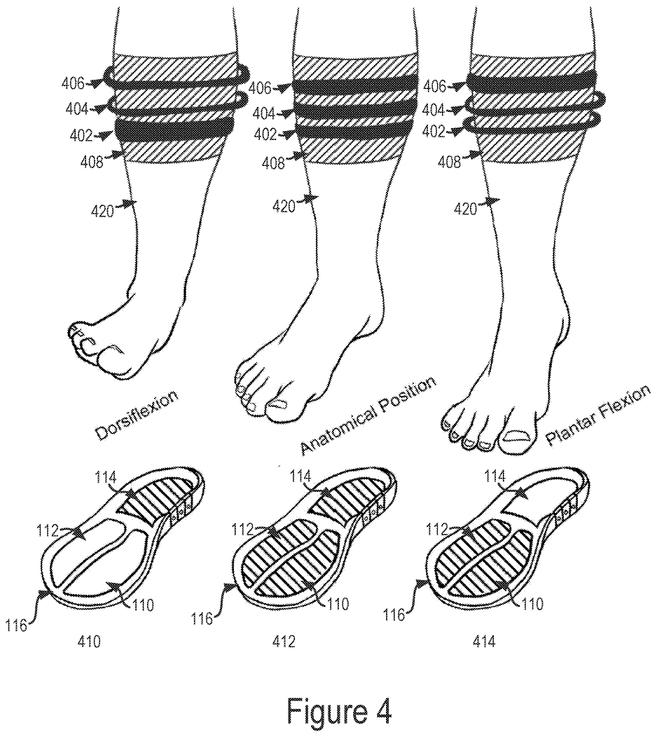

FIG. 4 shows an example at least one stimulation bladders as ring-shaped bladders. In particular, the at least one stimulation bladders can include a first ring-shaped stimulation bladder 402, a second ring-shaped stimulation bladder 404 and a third ring-shaped stimulation bladder 406. Each of the ring-shaped stimulation bladders can be structured such that when fluid flows into the bladder, the bladder provides a compressive force around the leg 420 of the user. One of the ring-shaped stimulation bladders can be fluidly coupled with one of the at least one sensor bladders. For example, the first ring-shaped stimulation bladder 402 can be fluidly coupled with the hindfoot sensor bladder 114, the second ring-shaped stimulation bladder 404 can be fluidly coupled with the medial forefoot sensor bladder 110 and the third ring-shaped stimulation bladder 406 can be fluidly coupled with the lateral forefoot sensor bladder 112. At least one conduit fluidly couple the at least one sensor bladders to the at least one ring-shaped stimulation bladders (the at least one conduit are not shown for simplicity). In various examples, the ring-shaped stimulation bladders 402, 404, and 406 can be wrapped around a leg-wrap 408, which can include a wearable leg-wrap with elastic lining. Also not shown in FIG. 4 is the one or more ports each stimulation bladder has to couple with the corresponding conduit. The medial forefoot sensor bladder 110, the lateral forefoot sensor bladder 112 and the hindfoot sensor bladder 114 are disposed over the shoe insert 116 in a manner described above in relation to FIG. 1. The shoe insert 116 can be positioned under the foot of the user and within a footwear worn by the user.

FIG. 4 shows example positions of the foot of the user. For example, FIG. 4 shows a first position 410 in which the foot of the user is in the dorsiflexion position, a second position 412 in which the foot of the user is in the anatomical position, i.e., the entire foot is resting on the shoe insert 116, and a third position 414 in which the foot is in the plantar flexion position. In the first position 410, the hindfoot sensor bladder 114 is pressed down on, which causes the fluid from the hindfoot sensor bladder 114 to flow into the first ring-shaped stimulation bladder 402, which in turn provides a compressive force in the area of the leg 420 under the first ring-shaped stimulation bladder 402. In the second position 412, the foot of the user is flat on the shoe insert 116 and presses down on all the sensor bladders. This causes the fluid from all the sensor bladders to flow into the corresponding stimulation bladders. For example, fluid from the medial forefoot sensor bladder 110 flows into the second ring-shaped stimulation bladder 404, and the fluid from the lateral forefoot sensor bladder 112 can flow into the third ring-shaped stimulation bladder 406. Each of the second ring-shaped stimulation bladder 404 and the third ring-shaped stimulation bladder 406, once inflated, can provide a compressive force in the respective area of the leg 420.

In the configuration shown in FIG. 4, the first ring-shaped stimulation bladder 402 is positioned closest to the foot, the third ring-shaped stimulation bladder 406 is positioned farthest from the foot, and the second ring-shaped stimulation bladder 404 is positioned between the first ring-shaped stimulation bladder 402 and the third ring-shaped stimulation bladder 406. However, these bladders can be positioned in any manner. The ring-shaped bladders can be spaced apart from each other with sufficient distance to allow the user to discern the difference in force from each of the bladders. In some examples, each ring-shaped stimulation bladder can be spaced apart from the adjacent ring-shaped stimulation bladder by at least 0.3 to 0.8 inches, or about 0.5 inches. In some instances, one of the ring-shaped stimulation bladder may by positioned on the user's thigh or any other part of the body where the user still has sense perception. FIGS. 1 and 4 provide examples of different configurations in which the stimulation bladders can be positioned on the user's leg or body. The number of ring-shaped stimulation bladders can be equal to the number of sensor bladders. In some examples, the two or more ring-shaped bladders can be fluidly coupled with a single sensor bladder, or a single ring-shaped stimulation bladder can be fluidly coupled with two or more sensor bladders. In the latter instance, the amount of force exerted on the user's body can vary based on the number of sensor bladders being pressed by the foot of the user. In some instances, a McKibben muscle can be utilized as the ring-shaped stimulation bladder. McKibben muscles are a type of pneumatic artificial muscles that reduce in length when inflated. In some examples, the McKibben muscles includes an inflatable inner tube/bladder inside a braided mesh, clamped at the ends. When the inner bladder is pressurized and expands, the geometry of the mesh acts like a scissor linkage and translates this radial expansion into linear contraction. This contraction can result in the compressive force imparted on the leg of the user. In some other instances, the ring-shaped stimulation bladder can be implement by a tube that is wrapped around the leg of the user, where when the tube is inflated, the expansion of the tube can impart a compressive force on the leg of the user. As another example, other pneumatic or hydraulic (where the fluid is a liquid) artificial muscles can be utilized.

In some examples, the ring-shaped stimulation bladder may or may not wrap around the entire circumference of the leg or the portion of the body where the bladder is positioned. In some instances, the ring-shaped stimulation bladder is wrapped around more than half the circumference of the part of the body at which the stimulation bladder is positioned. That is, more than half the circumference of the part of the body around which the ring-shaped stimulation bladder is wrapped is imparted with the compressive force during inflation.

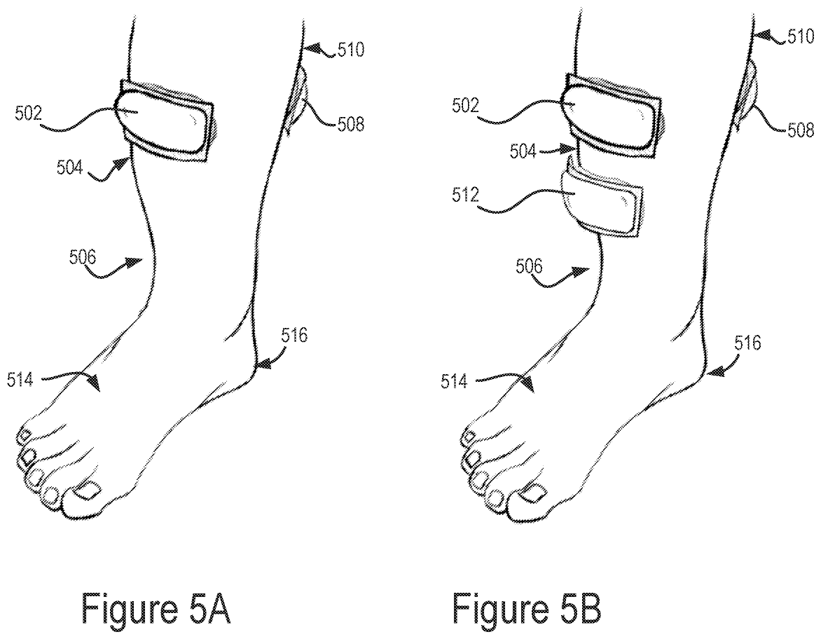

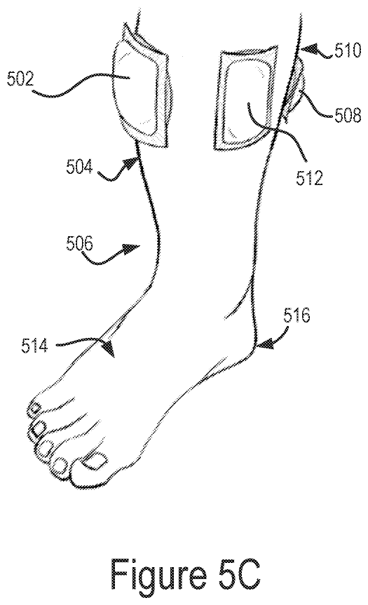

FIGS. 5A-5C show various positions for one or more stimulation bladders. In particular, FIG. 5A shows a first stimulation bladder 502 positioned on the front portion (e.g., near the shin 504) of the leg 506 of the user and a second stimulation bladder 508 positioned near the back portion (e.g., the calf) of the leg 506 of the user. The first stimulation bladder 502 and the second stimulation bladder 508 can be positioned at approximately the same distance from the base 516 of the foot 514. In some instances, the first stimulation bladder 502 and the second stimulation bladder 508 can be positioned at different distances from the base 516 of the foot 514. FIG. 5B shows the first stimulation bladder 502 and the second stimulation bladder 508 as shown in FIG. 5A, but also shows a third stimulation bladder 512 also positioned near the shin 504 of the leg 506, but positioned below or inferior to the first stimulation bladder 502. The third stimulation bladder 512 may be positioned as low as to the ankle of the leg 506. Generally, the stimulation bladders shown in FIG. 5B can be positioned on the leg of the user at different distances from the base 516 of the foot 514 of the user. In some instances the first stimulation bladder 502 and the third stimulation bladder 512 can be aligned along a axis, but in some other instances, the first stimulation bladder 502 and 512 may not be aligned. FIG. 5C shows an example where a plurality of stimulation bladders are positioned on the leg of the user at approximately the same distance from the base of the foot. The stimulation bladders shown in FIG. 5C are not aligned along the vertical axis. In some instances, the stimulation bladders can be positioned at approximately the same distance from each other around the leg 506 of the user. The configuration shown in FIG. 5C can be similar to that shown in FIG. 1.

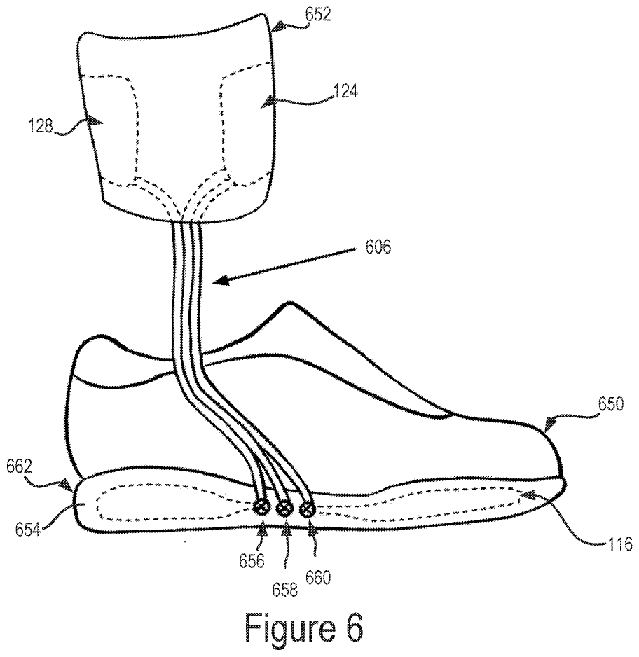

FIG. 6 shows a portion of an example soft robotics system 600 where at least one conduit that fluidly couples at least one sensor bladder with at least one stimulation bladder is coupled with a shoe of the user. For example, the soft robotics system 600 can include the at least one conduit 606 that can fluidly couple the at least one sensor bladder 102 to the lateral forefoot stimulation bladder 124, the hindfoot stimulation bladder 128, and/or the medial forefoot stimulation bladder 126 (not shown). As described, the at least one sensor bladder 102 can include one or more appropriate sensor bladders described in this disclosure that can fit within a shoe or a sock or an insert, which can fluidly be connected to the respective stimulation bladders via the conduit 606. The stimulation bladders in the example soft robotics system 600 can be secured by a housing 652 that can attach to a user's body. For example, the housing 652 can include a strap or a sleeve of some sort that can be fitted around the user's leg, knee, or thigh. The soft robotics system 600 is similar to the soft robotics system 100 discussed above in relation to FIG. 1 in that similar to the soft robotics system 100, the soft robotics system 600 also includes the shoe insert 116 having at least one sensor bladder, includes at least one stimulation bladder and includes at least one conduit providing fluid coupling between the at least one sensor bladder and at least one stimulation bladder. However, unlike the soft robotics system 100, in which the at least one conduit 106 is routed from between the user's foot and the shoe, the at least one conduit 606 of the soft robotics system 600 is routed from outside the shoe 650. The sole 662 of the shoe 650 can include a plurality of openings that can provide access to the conduit ports 120 on the shoe insert 116. Specifically, a sidewall 654 of the sole 662 can define at least one opening: a first opening 656, a second opening 658, and a third opening 660 that can allow ends of the at least one conduit 606 to couple with the three conduit ports 120 of the shoe insert 116. In some instances, the sidewall 654 may define a single opening that can allow all of the conduits to pass through and couple with the conduit ports 120 on the shoe insert 116. In some instances, where there is not shoe insert 116 and the at least one sensor bladder is coupled with a sock or is otherwise positioned between the foot and the sole of the shoe, the at least one conduit can be allowed to pass through the at least one opening in the sidewall 654 and couple directly with the at least one sensor bladder. The at least one opening need not necessarily be positioned on the sidewall 654 of the sole 662 and can be positioned at any other location on the sole 662 or any other location on the shoe 650. For example, the at least one opening can be positioned on the upper portion (above the sloe 662) of the shoe 650. In some instances, the sidewall 654 or other portions of the shoe 650 can include more than one opening that are positioned in different locations. For example, one or more of the at least one opening can be positioned on the sidewall of the sole 662 opposite to the sidewall 654. In some other instances, the at least one opening can be positioned at different locations on the upper portions of the shoe 650. Providing different locations can allow routing the at least one conduit directly to the at least one sensor bladder.

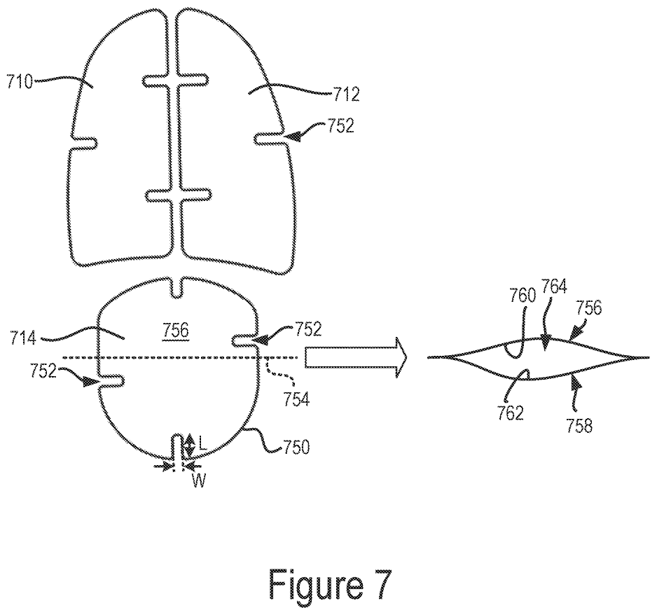

FIG. 7 shows an example configuration of at least one sensor bladder. In particular, FIG. 7 shows top views of a medial forefoot sensor bladder 710, a lateral forefoot sensor bladder 712, and a hindfoot sensor bladder 714, which can be used to implement the medial forefoot sensor bladder 110, the lateral forefoot sensor bladder 112, and the hindfoot sensor bladder 114 shown in FIGS. 1, 2 and 4, and can also be used to implement the sensor bladders shown in FIGS. 3A and 3B. FIG. 7 also shows a cross-sectional view of the hindfoot sensor bladder 714 along the axis 754. The cross-sectional view for the hindfoot sensor bladder 714 can be representative of the cross-sectional structure of the medial forefoot sensor bladder 710 and the lateral forefoot sensor bladder 712.

The hindfoot sensor bladder 714 has a perimeter 750. The perimeter 750 can have a shape that can conform to the shape of the hindfoot of the user. The hindfoot sensor bladder 714 includes a first membrane 756 and a second membrane 758 that terminate at the perimeter 750 of the hindfoot sensor bladder 714. The first membrane 756 defines a first inner surface 760 while the second membrane 758 defines a second inner surface 762. The first inner surface 760 and the second inner surface 762 define a volume 764 of the hindfoot sensor bladder 714. While not shown in FIG. 7, the hindfoot sensor bladder 714 includes a port that opens into the volume 764. The port can be defined in one of the first membrane 756 or the second membrane 758, or can be positioned between the first membrane 756 and the second membrane 758 along the perimeter 750 of the hindfoot sensor bladder 714. In some instances, an adhesive can be utilized to ensure that there are not gaps between the port and the membranes of the hindfoot sensor bladder 714 and ensure that the coupling of outside of the port with the membranes is air tight (or generally fluid-tight). The hindfoot sensor bladder 714 can include walls that extend between the first inner surface 760 and the second inner surface 762. The walls 752 positioned near the perimeter 750 and extending inward form the perimeter 750 are examples of such walls. As discussed herein, the walls formed within the sensor bladders can include a barrier that extends between the first inner surface 760 and the second inner surface 762 or can include a bonding of the first inner surface 760 with the second inner surface 762. That is, a portion of the first inner surface 760 can be bonded with the opposite portion of the second inner surface 762 to create a wall-like structure. The walls 752 are formed to ensure that the bladder has a contiguous volume. That is, the walls are limited to not divide the volume 764 into separate volumes. The inclusion of the walls 752 that extend inward from the perimeter can help in providing tensional forces to restrict the vertical profile of the bladder but also provide a volume 764 that has low resistance to inflow of fluid. As a result, fluid can easily reenter the hindfoot sensor bladder 714 when the user is no longer pressing down in the hindfoot sensor bladder 714. The walls 752 that extend inward from the perimeter 750 can form one or more slots as part of the sensor bladders 710, 712, and/or 714, in which the one or more slots can be substantially rectangular in shape. The size of each of the one or more slots may be equal or different based on user foot size and/or shape, gait pattern, fluid dynamics, among others. As described above, the walls 762 can be configured to restrict bladder expansion and be used to modify inflated bladder thickness as well as steady state displacement of fluid.

The walls 752 can extend inwards from the perimeter 750 only to the extent that they do not make contact with the perimeter 750 at another location. For example, the walls 752 shown above and below the axis 754 do not extend all the way across to the opposite end of the hindfoot sensor bladder 714. In some examples, the walls 752 can extend inwards no more than half the distance between a first location on the perimeter 750 where the walls 752 are positioned and a second location on the perimeter 750 positioned opposite the first location.

In some examples, the walls 752 can be evenly distributed along the perimeter 750 of the bladder. In some other examples, the width W of the walls 752 can be less than the length L of the walls 752, where the width can be considered as a distance between two locations on the perimeter where a wall extends inwards, and a length of the wall can be considered as the distance by which the wall normally extends inwards from those two locations. While the walls 752 have been shown in relation to specific sensor bladders, in particular the medial forefoot sensor bladder 710, the lateral forefoot sensor bladder 712 and the hindfoot sensor bladder 714, it should be understood that the walls 752 can be incorporated any other type of sensor bladder such as, for example, the single forefoot sensor bladder 302 shown in FIG. 3A and the anterior forefoot sensor bladder 306 and the posterior forefoot sensor bladder 308 shown in FIG. 3B.

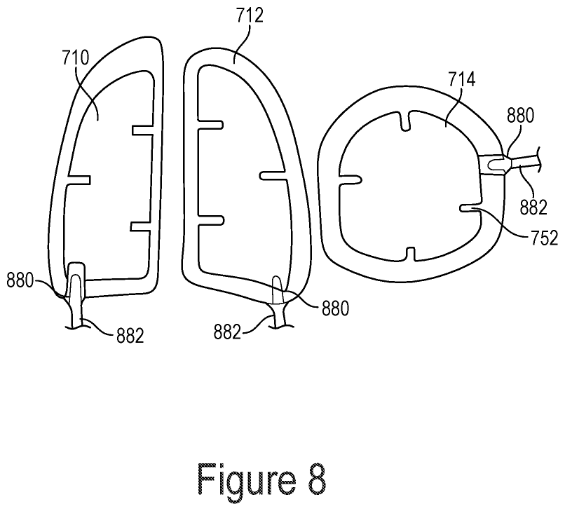

FIG. 8 shows a photograph depicting top views of manufactured sensor bladders. Specifically, FIG. 8 shows photographs of the manufactured in the manner discussed above in in relation to FIG. 7. Each of the medial forefoot sensor bladder 710, the lateral forefoot sensor bladder 712 and the hindfoot sensor bladder 714 includes one or more walls 752 that extend inward from the outer perimeter. Each bladder also includes a port 880 that allows coupling with a conduit 882.

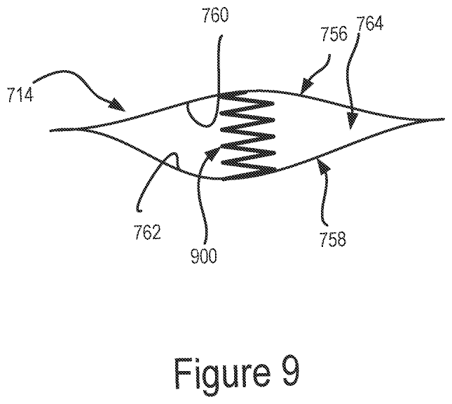

FIG. 9 shows an example at least one sensor bladder with an expander. In particular, the hindfoot sensor bladder 714 that has an expander 900 positioned between the first inner surface 760 and the second inner surface 762. The expander 900 can be a spring, elastic materials, foam or anything that returns to an original shape after compression. While FIG. 9 depicts the expander 900 in relation to only the hindfoot sensor bladder 714, the expander 900 can be similarly implemented for other sensor bladders such as medial forefoot sensor bladder 710 and lateral forefoot sensor bladder 712. The expander 900 can be configured to compress responsive to compressive force from the foot of the user and to expand and draw fluid back into the hindfoot sensor bladder 714 responsive to the reduction in compressive force from the foot. The expander 900 shown in FIG. 9 can be a coiled spring. However, other types of springs can also be used such as, for example, flat springs, disk springs, etc. In some examples, the expander 900 can include materials such as silicone sponge or foam to help expansion of the sensor bladder and draw fluid back into the sensor bladder. While not shown in the figures, in some instances where there is a need to draw fluid into the stimulation bladders, expander 900 can be incorporated into the stimulation bladders.

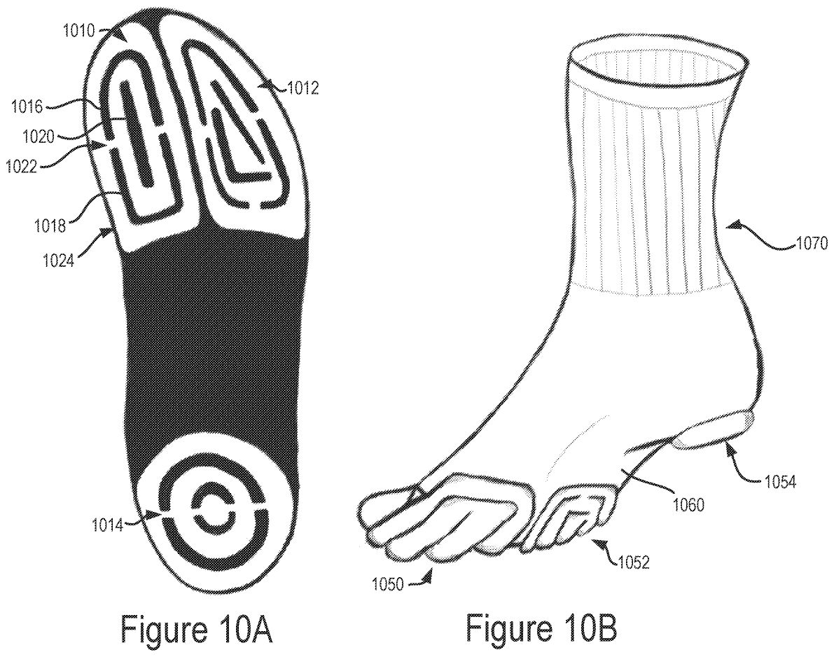

FIGS. 10A and 10B show another example configuration of at least one sensor bladder. In particular, FIG. 10A shows sensor bladders including walls that are positioned within the perimeter of the sensor bladder. FIG. 10B shows the sensor bladder of FIG. 10A attached to a sock. Referring to FIG. 10A, the at least one sensor bladder includes a medial forefoot sensor bladder 1010, a lateral forefoot sensor bladder 1012, and hindfoot sensor bladder 1014. The medial forefoot sensor bladder 1010 includes a set of walls: a first wall 1016, a second wall 1018, and a third wall 1020. The set of walls are positioned and shaped in a manner that can be similar to the shape of the perimeter 1024 of the medial forefoot sensor bladder 1010. For example, the first wall 1016 and the second wall 1018 collectively are shaped similar to the shape of the perimeter 1024. The first wall 1016 and the 1018 are positioned offset from the perimeter 1024 such that they do not make contact with the perimeter 1024. However, in some instances, walls can make contact with the perimeter 1024. The first wall 1016 and the second wall 1018 can be separated by at least one gap 1022. Inclusion of the at least one gap 1022 ensures that the set of walls do not separate the medial forefoot sensor bladder 1010 into individual chambers that are completely walled off, i.e., no fluid can flow between them. Instead, despite the presence of the set of walls, the medial forefoot sensor bladder 1010 forms a contiguous volume. The third wall 1020 can be positioned within the area enclosed by the first wall 1016 and the second wall 1018.

The shapes and sizes of the walls within the at least one sensor bladder can vary based on the implementation. The inclusion of the walls helps in reducing the vertical profile of the sensor bladder. Reducing the vertical profile of the bladder in turn helps in reducing user discomfort. The positioning of the walls therefore can be dependent on the shape and size of the sensor bladder. For example, the set of wall in the medial forefoot sensor bladder 1010 can be positioned differently from the set of walls positioned in the lateral forefoot sensor bladder 1012 or the hindfoot sensor bladder 1014.

FIG. 10B shows at least one sensor bladder attached to a sock. In particular, FIG. 10B shows portions of a bottom surface 1060 of the sock 1070 that has a hindfoot sensor bladder 1054, a medial forefoot sensor bladder 1050, and a lateral forefoot sensor bladder 1052 attached to a bottom surface 1060 of the sock 1070. The medial forefoot sensor bladder 1050, the lateral forefoot sensor bladder 1052, and the hindfoot sensor bladder 1014 can be similar to the medial forefoot sensor bladder 1010, the lateral forefoot sensor bladder 1012 and the hindfoot sensor bladder 1014, respectively, shown in FIG. 10A. Attaching the at least one sensor bladder to the sock 1070 can improve the ease with which the user can incorporate the sensor bladders into the footwear. For example, in some instances, it may be difficult to position an insole into the footwear. In some such instances, wearing a sock that has the sensor bladders installed on the bottom surface can be beneficial.

In some instances, the sensor bladders can be positioned over both the shoe insole and the socks. That is at least one sensor bladder can be positioned on the insole, while at least one additional sensor bladder can be positioned on the sock. The sensor bladders on the insole and the sock can be in fluid communication with the corresponding stimulation bladders, which can be adhered to the leg of the user based on various aspects described herein. In some instances, one foot of the user can have the at least one sensor bladder positioned on the insole and the other foot of the user can have the at least one sensor bladder positioned on a sock worn on that leg. Sensor and stimulation bladders that are in fluid communication may or may not be positioned on the same leg. For example, a sensor bladder may be positioned on the foot of one leg, while the stimulation bladder can be positioned on the other leg, or for that matter anywhere on the body of the user.

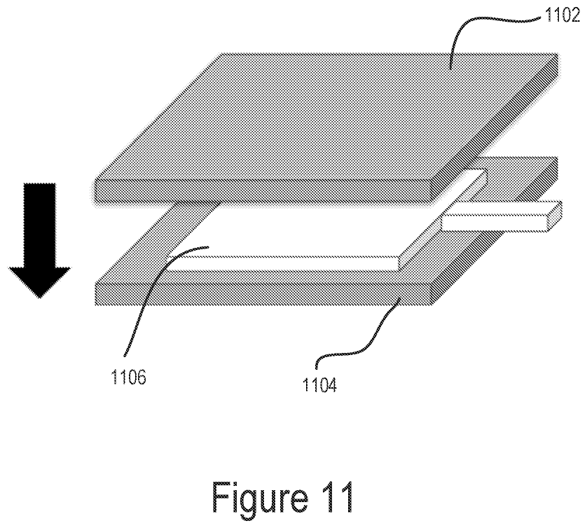

FIG. 11 shows an example structure of the one or more bladders discussed herein. A bladder can be formed by attaching a first membrane 1102 to a second membrane 1104 with a blocking material 1106 positioned therebetween. The first membrane 1102 and the second membrane 1104 are attached to each other at locations that are not covered by the blocking material 1106. In the example shown in FIG. 11, the first membrane 1102 and the second membrane 1104 can attached to each other along the peripheries, while remaining un-attached at the center portion covered by the blocking material 1106. The center portion can form the expanding volume of the bladder. In some instances, the first membrane 1102 and the second membrane 1104 can be attached to each other using various means such as adhesive, stitches, rivets, staples, etc. that can provide an air-tight seal for the operating pressure of the bladder. The blocking material blocking material 1106 can be patterned to form walls within the bladder. For example, the blocking material 1106 can have a pattern where the blocking material 1106 is lacking in positions where a wall is desired such that the opposing membranes will attach to each other in those positions and form a wall. The blocking material 1106 can be patterned for example to form the pattern of walls discussed herein.

Other approaches for forming the bladder can also be used. For example, the bladder can be formed using a mold for heating the material used for forming the membranes of the bladder. In some other examples, 3D printing can be used to form the bladders.

In some examples, a thermoplastic polyurethane (TPU) film can be utilized for forming the membranes of the bladders (both the sensor and the stimulation bladders). In some instances, woven, non-stretch TPU with nylon coating can be used. In some instances, a bi-directional, high-stretch knitted fabric can be used. In some other instances, elastic thermoplastics could be utilized for forming the membranes of the bladders and the conduits. The blocking material 1106 can be any material that does not bond with the material of the membranes (such as for example, when the membranes are heated to form a bond). In some examples, the blocking material 1106 can include paper, glassine, plastic, metal, Teflon etc.

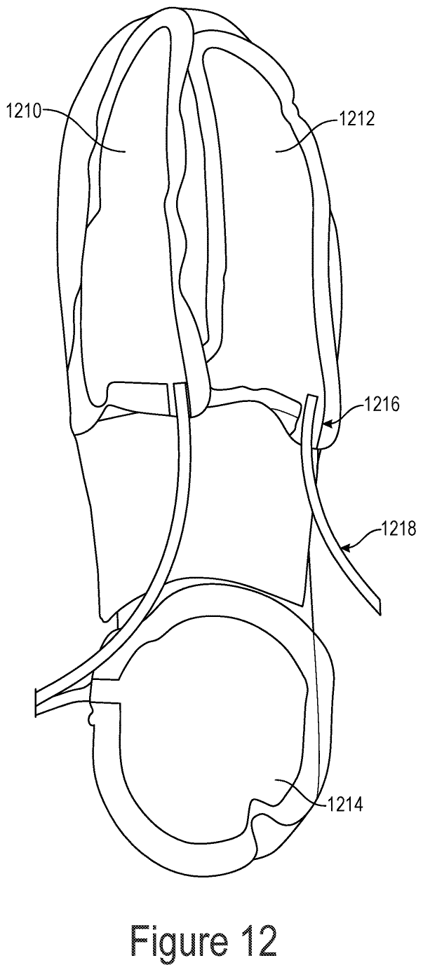

FIG. 12 shows a photograph of an example set of sensor bladders. In particular, FIG. 12 shows sensor bladders formed using TPU film. The configuration of the sensor bladders shown in FIG. 12 is similar to that shown in FIG. 1, in that FIG. 12 slow shows a medial forefoot sensor bladder 1210, a lateral forefoot sensor bladder 1212, and a hindfoot sensor bladder 1214. While the sensor bladders shown in FIG. 12 do not include any walls, such as those shown in FIGS. 7, 10A, and 10B, these bladders can be manufactured to include such walls. Each sensor bladder includes at least one port that allows fluid to flow to and from the bladder. For example, the lateral forefoot sensor bladder 1212 includes a port 1216, which is coupled with a conduit 1218. Similar ports are provided for each of the other sensor bladders.

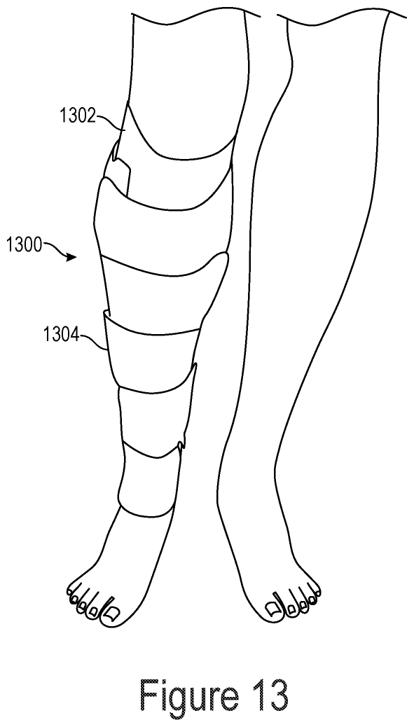

FIG. 13 shows an example stimulation bladder housing attached to a user's leg. The stimulation bladder housing 1300 can include an inner layer (not shown) and an outer layer 1304. In the example shown in FIG. 13, the inner layer can include multiple patches that are attached to the inner surface of the outer layer 1304 to form pockets within each of which the stimulation bladder 1306 is positioned. The outer layer 1304 is wrapped over the leg of the user. In some examples, the inner layer can be an expandable mesh. The outer layer 1304 can be relatively rigid in comparison with the inner layer. When the stimulation bladder is inflated, the outer layer 1304 can maintain a stable perimeter while the inner layer 1302 can expand with the bladder into the leg.

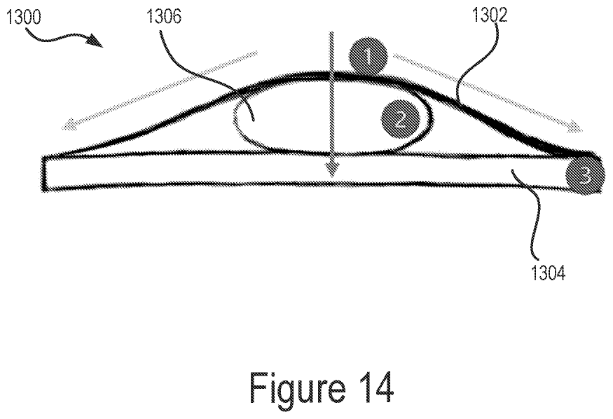

FIG. 14 shows a portion of a cross-sectional view of the example stimulation bladder housing 1300 discussed above in relation to FIG. 13. A stimulation bladder 1306 is positioned between the inner layer 1302 and the outer layer 1304. The rigid and inelastic outer layer 1304 can be wrapped around the leg without applying any undue compression. The inner layer 1302, which can be elastic, can normally apply a compressive force on the stimulation bladder 1306. This ensures that the elastic inner layer 1302 does not apply any compressive force to the leg. The compressive force on the stimulation bladder 1306 also ensures that when the stimulation bladder 1306 is not inflated by the fluid from the corresponding sensor bladder, fluid in the stimulation bladder 1306 is forced back into the corresponding sensor bladder. When the stimulation bladder 1306 is inflated, the inner layer 1302 can stretch and allow the stimulation bladder 1306 to impart pressure on the leg of the user.

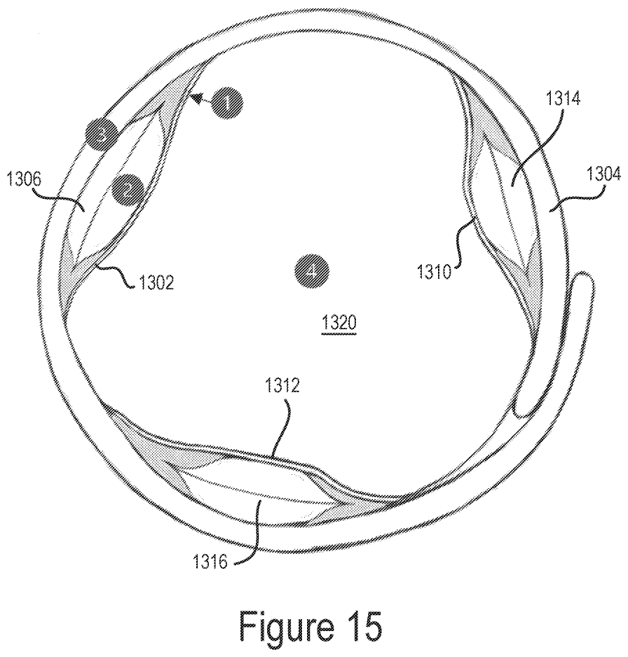

FIG. 15 shows a cross-sectional view of the example stimulation bladder housing 1300 discussed above in relation to FIG. 13. The stimulation bladder housing 1300 can include a first inner layer 1302, a second inner layer 1310, and a third inner layer 1312, each of which is attached to the inner surface of the outer layer 1304. The first stimulation bladder 1306 is positioned between the first inner layer 1302, a second stimulation bladder 1314 can be positioned between the second inner layer 1310 and the outer layer 1304, and a third stimulation bladder 1316 can be positioned between the third inner layer 1312 and the outer layer 1304. The outer layer 1304 can be removably wrapped around the leg 1320 of the user, and can be held in place by a belt, Velcro, or other binding means.

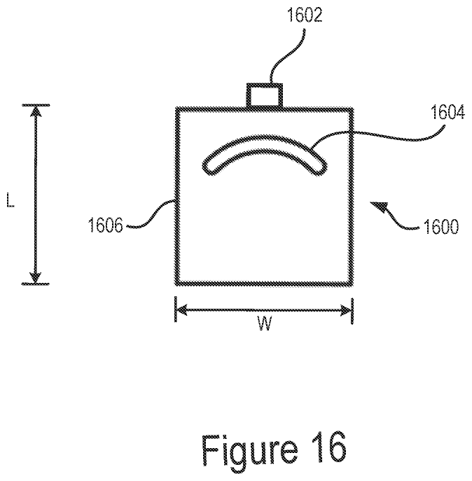

FIG. 16 shows a top view of an example stimulation bladder. The stimulation bladder 1600 can be used to implement stimulation bladders discussed herein such as, for example, those discussed in relation to FIGS. 1, 2, 5A-5C, 6, 13-15. The stimulation bladder 1600 has a perimeter 1606 to which a port 1602 is attached. The port 1602 allows fluid to flow into and out of the stimulation bladder 1600. A wall 1604 can be positioned in front of the port 1602, which can disrupt the flow of fluid into the bladder as well as increase the compressive forces in the stimulation bladder 1600. As discussed above, it is desirable that the fluid be returned to the sensor bladder once the sensor bladder is decompressed or is not activated. One approach to achieve this goal is to increase the compressive forces on the stimulation bladder such that when the corresponding sensor bladder is deactivated, the fluid in the stimulation bladder can flow back into the sensor bladder. Additionally, in some instances, it may be advantageous to disrupt the fluid flow into the stimulation bladder itself. The inclusion of the wall 1604 can facilitate both these approaches. The wall 1604 can be positioned no more than half the length L of the stimulation bladder 1600 away from the perimeter 1606 where the port 1602 is positioned. The wall 1604 can have a curved shape where the convex side of the curve faces the port 1602, however, in some other examples, the wall 1604 can be liner instead. In some instances, the wall 1604 can be discontinuous. That is, the wall 1604 can include channels that allow fluid to flow though. The size of the discontinuities can be adjusted based on the degree of flow resistance needed.

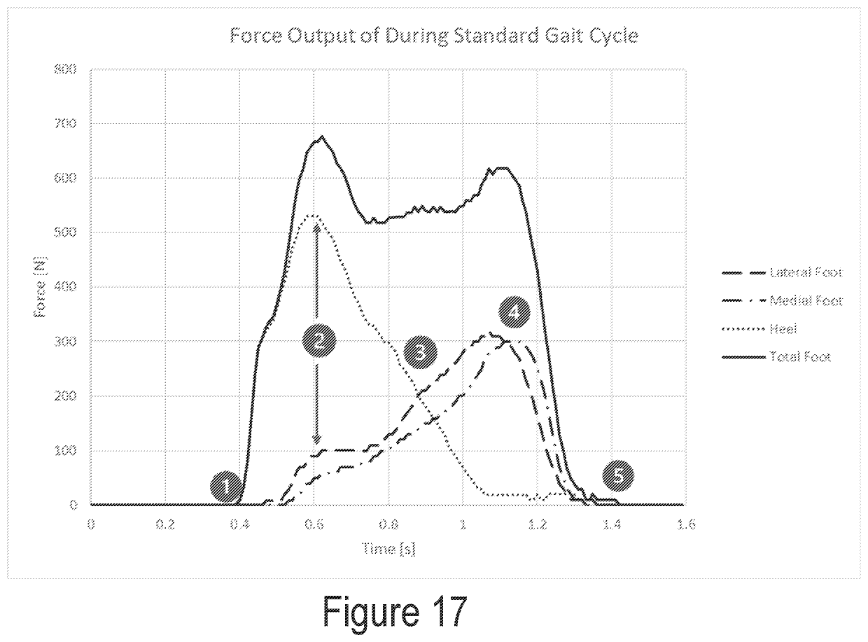

FIG. 17 shows graphical representation of forces measured at the various sensor bladders of an example soft robotics system. In particular, FIG. 17 shows the magnitude of force sensed at a medial forefoot sensor bladder, a lateral forefoot sensor bladder, a hindfoot sensor bladder, and the total force which is the sum of the forces measured at these sensor bladders. The magnitude of the forces measured at each bladder is measured during various phases of the gait cycle. For example, the phase 1 represents the heel strike, which is the first contact that the foot makes with the ground. During heel strike, the heel acts as a roller and enables the body to load against the ground and roll or translate toward placing pressure on the forefoot. During this phase, the hindfoot (heel) sensor bladder deflates and the corresponding stimulation bladder inflates. Phase 2 represents a transitional phase when the foot rolls on the heel and the forefoot makes contact with the ground. In this phase, the foot is loaded on the lateral or outside and translates towards the toes until the foot is flat on the ground. During this phase, all the underfoot sensor bladders deflate and consequently all the corresponding stimulation bladders also inflate.

Phase 3 represents the heel lift stage. This is a transitional stage for moving the body's weight from flat foot onto the toes. During this phase, the heel is lifted off the ground and the weight gradually moves to the toes. In this phase, the medial and lateral forefoot sensor bladders deflate causing the corresponding stimulation bladders to inflate. In addition, the hindfoot sensor bladder inflates and the corresponding stimulation bladder deflates. Phase 4 represents the toe-off stage when all of the body's weight is on the toes and the toes are used to push the foot off the ground, moving the body forward. In this stage, maximal forces are applied to the forefoot. As a result, the medial and lateral forefoot sensor bladders deflate, and the corresponding stimulation bladders inflate. Phase 5 represents the lift or swing phase, which is the last phase before repeating the gait cycle. In this phase, the foot comes off the ground and the fluids are relocated into the underfoot sensor bladders. As a result, the medial and lateral forefoot sensor bladders and the hindfoot sensor bladder inflate.

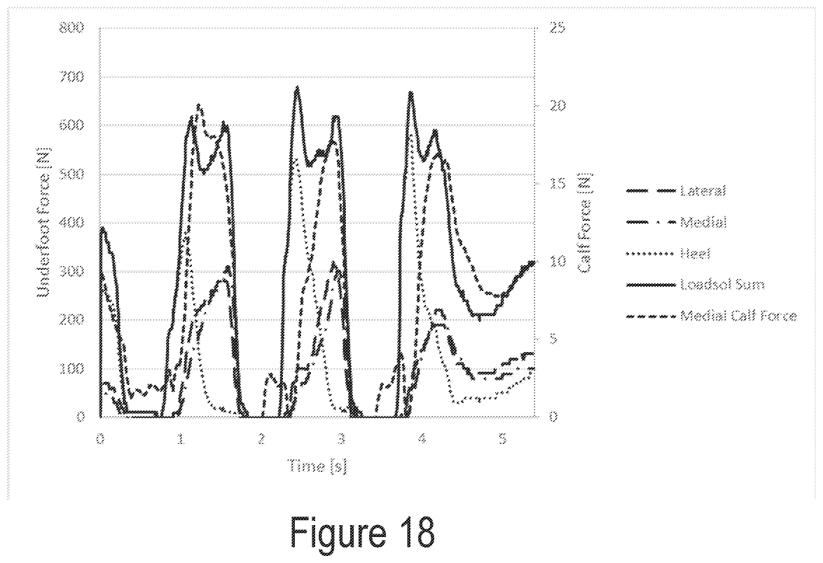

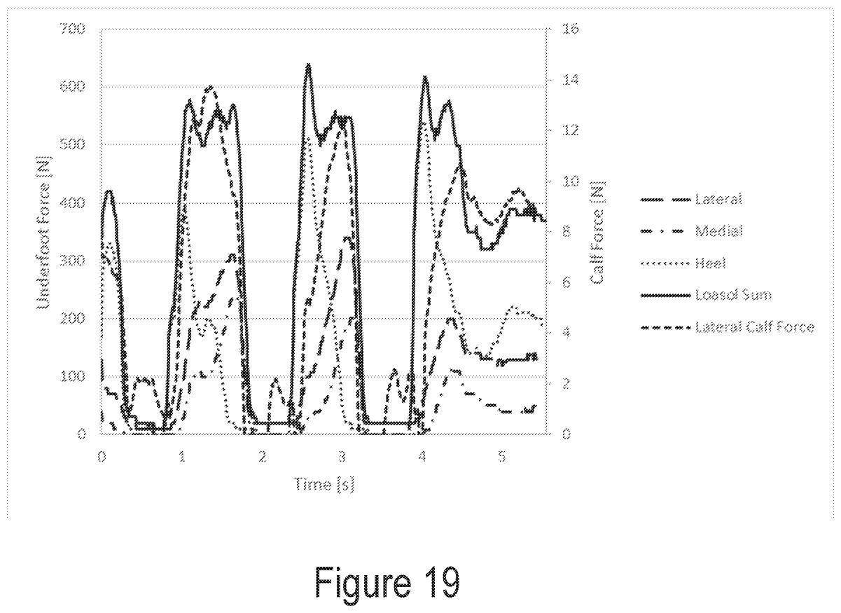

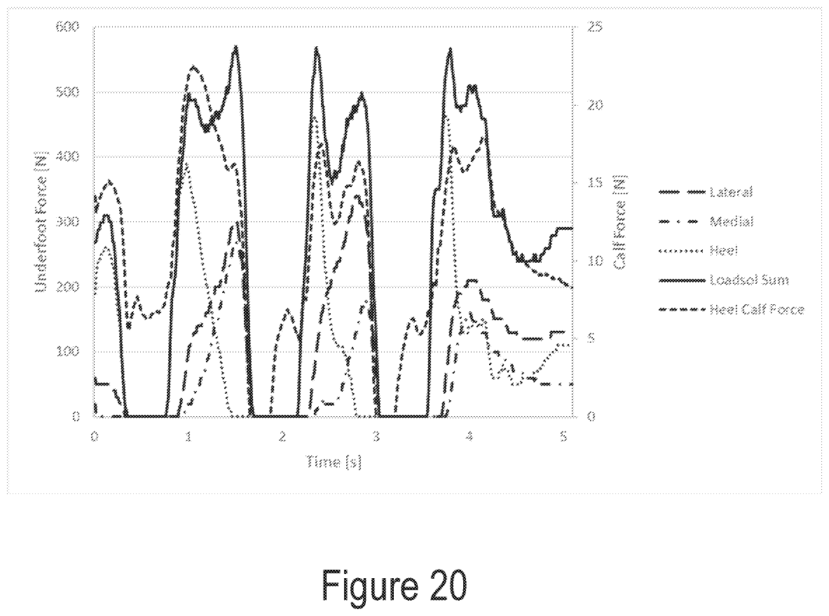

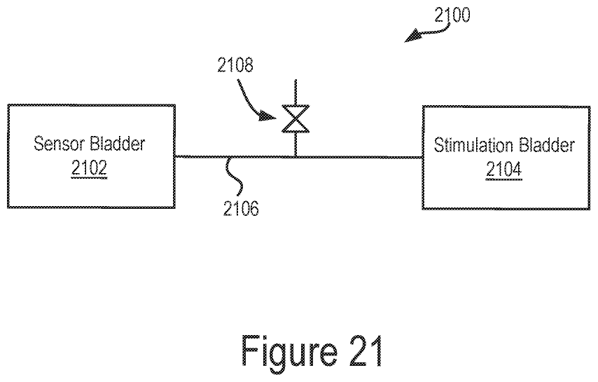

FIGS. 18-20 show additional graphical representations of forces measured at various sensor and stimulation bladders. For example, FIG. 18 shows the forces measured at various sensor bladders during multiple gait cycles as well as the force measured at the stimulation bladder corresponding to the medial forefoot sensor bladder, FIG. 19 shows the forces measured at various sensor bladders during multiple gait cycles as well as the force measured at the stimulation bladder corresponding to the lateral forefoot sensor bladder, and FIG. 20 shows the forces measured at various sensor bladders during multiple gait cycles as well as the force measured at the stimulation bladder corresponding to the hindfoot sensor bladder.

Table 1 below depicts the analysis of the performance of an example soft robotics system.

TABLE 1

| | AVG. | Max | | |
|---|---|---|---|---|
| | Stimulation | Stimulation | Response | Exchange |
| | Force when | Force when | Time | Time |
| Bladder Network | Standing [N] | Walking [N] | [ms] | [ms] |
| Hindfoot | 15.31 | 17.49 | 40 | 210 |
| Medial Forefoot | 6.73 | 17.74 | 10 | 530 |
| Lateral Forefoot | 5.54 | 12.49 | 40 | 560 |
| No Bladder | 0.00 | 4.69 | N/A | N/A |

Table 1 shows the measured force magnitudes at the sensor and stimulation bladders and the response time for the stimulation bladder to provide a threshold force in response to the deflation of the corresponding sensor bladder. The bladder network indicates the sensor-stimulation bladder system for which measurements were made. For example, the hindfoot bladder network represents the hindfoot sensor bladder and the corresponding stimulation bladder and the conduit that couples the two bladders. Similarly, the medial forefoot bladder network represents the medial forefoot sensor bladder and the corresponding stimulation bladder and the conduit, and the lateral forefoot bladder network represents the lateral forefoot sensor bladder and the corresponding stimulation bladder and the conduit. The average stimulation force when standing was measured at the stimulation bladder when the subject stood still for 10 seconds. The average of all the stimulation bladder force measurements over the 10 seconds were averaged. The maximum stimulation force when walking was based on the middle gait cycle phase form each data set. The maximum value for the force measured at the stimulation bladder during the middle gait was determined. The response time was also measured form the middle gait cycle. The response time value is the difference in time from when the force at the sensor bladder begins to increase to when the force at the stimulation bladder begins to increase. The exchange time is also measured during the middle gate phase. The exchange time represents the difference in time from when the force at the stimulation bladder begins to increase to when the force at the stimulation bladder reaches its maximum value.

The force values at the sensor and the stimulation bladders was measured using force sensors such as for example Novel loadsol (MPL 3 zone) and Novel loadpad (MPL high tech, 11×5 cm). In particular, the loadsol was used to measure the force under the sensor bladders while the loadpad was used to measure the force under the stimulation bladder. Each bladder network was pressurized with air (although other fluids could also be used). In some instances, a combination of air and liquids can also be used as the fluid.

FIG. 21 shows a schematic of an example bladder system. The bladder system 2100 can represent any of the bladder systems discussed above (e.g., the hindfoot sensor bladder 114 and the hindfoot stimulation bladder 128 shown in FIG. 2). The bladder system 2100 includes a sensor bladder 2102, a conduit 2106 and a stimulation bladder 2104. While only a single sensor bladder and a single stimulation bladder is shown in FIG. 21, a plurality of sensor bladders can be used, or a plurality of stimulation bladders can be used, in some instances, one sensor bladder can be coupled with a plurality of stimulation bladders and in some other instances a plurality of stimulation bladders can be coupled with a single sensor bladder. Also, while a single conduit is shown, the number of conduits can depend on the number of sensor or stimulation bladders or a network of conduits can be utilized to fluidly couple the sensor bladders to the stimulation bladders. The conduit 2106 fluidly couples the sensor bladder 2102 with the stimulation bladder 2104. Before operation, the bladder system 2100 is pressurized with the working fluid. For example, a valve 2108 can be coupled with the conduit 2106 to introduce the fluid into the bladder system 2100 at the desired pressure. Once the desired pressure is achieved, the valve 2108 can be closed and maintained closed during operation. The valve 2108 can be used to re-introduce fluid into the bladder system 2100 to compensate for any fluid leakages. Based on the activation or pressurization of the sensor bladder 2102 the fluid can move back and forth between the sensor bladder 2102 and the stimulation bladder 2104 via the conduit 2106.

In some instances, the sensor bladders discussed herein can be attached to a sock instead of an insole. For example, a sock can have an inner surface and an outer surface. The at least one sensor bladders can be attached to the inner surface, the outer surface or both the inner surface and the outer surface. In some examples, the sock can have at least two layers, where at least one sensor bladder is positioned between the two adjacent layers. This can help prevent the sensor bladder from making direct contact with the skin of the user and can also protect the bladder from debris or other harsh surfaces in the footwear. In some examples, the sock can include an upper portion that can extend beyond the ankle of the foot. The stimulation bladders discussed herein can be positioned on the upper portion. Again, in a manner similar to the sensor bladders discussed above, the stimulation bladders can be attached to an inner surface, an outer surface, or both the inner surface and the outer surface of the upper portion of the sock. In some instances, both the sensor bladders and the stimulation bladders can be attached to the sock. In some other instances, either the sensor bladders or the stimulation bladders, but not both, can be attached to the sock. The sock can be combined with any of the examples discussed herein. As an example, the sensor bladders can be attached to an insole of the footwear but the stimulation bladders can be attached to the sock. At least a portion of the conduits can also be attached to the sock to securely route them to the ports on the stimulation bladders. In some instances, the sensor bladders can be attached to the sock, while the stimulation bladders can be directly attached to the leg of the user or can be attached to a leg-wrap.

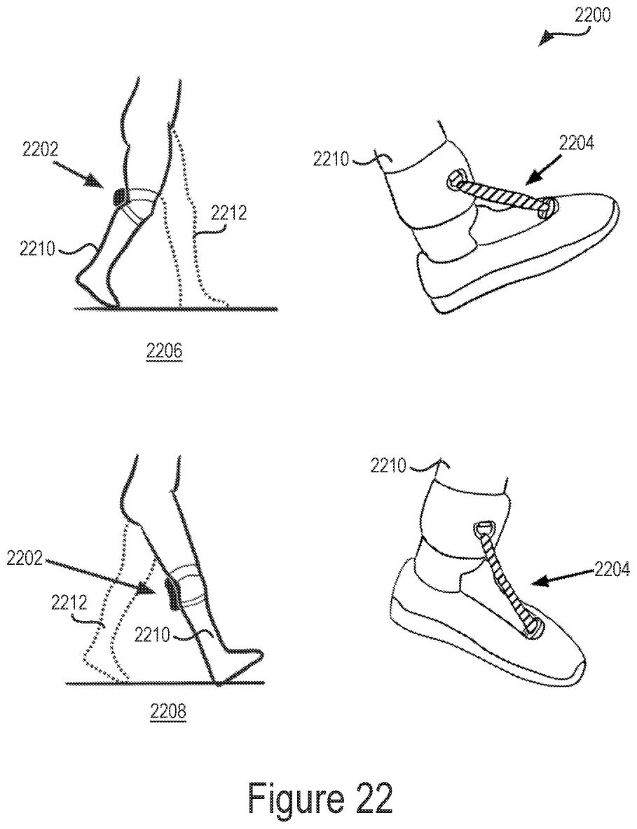

FIG. 22 shows schematics of another example soft robotics system 2200. In particular, the soft robotics system 2200 can be used to address drop-foot ailments in users. Individuals suffering from drop-foot can have difficulty in lifting the front part of the foot. This may result in dragging the foot during walking. The soft robotics system 2200 shown in FIG. 22 can address the drop-foot ailment in users with a sensor bladder and actuation bladder. For example, the soft robotics system 2200 can include a sensor bladder 2202 and an actuation bladder 2204. While FIG. 22 does not show a conduit, at least one conduit can fluidly couple the sensor bladder 2202 with the actuation bladder 2204. The actuation bladder can be configured to contract in length in response to pressurization. For example, when the sensor bladder 2202 is compressed, the fluid from the sensor bladder 2202 can flow into the actuation bladder 2204 via the at least one conduit. The pressurization of the actuation bladder 2204 can cause the length of the actuation bladder 2204 to decrease. One end of the actuation bladder 2204 is attached to the top of the foot while the other end is attached to the leg. The compression of the actuation bladder 2204 causes the toe of the foot to be lifted, thereby mitigating the drop-foot ailment of the user.

FIG. 22 shows two positions in the gait of the user. The first position 2206 shows the swing-lift position of the right leg 2210, where the right foot is about to be lifted off the ground. Prior to this position, the sensor bladder 2202, which is positioned behind the knee of the user, is compressed because of the bending of the leg at the knee. This compression causes the fluid from the sensor bladder 2202 to flow into the actuation bladder 2204 attached to the foot. The flow of fluid into the actuation bladder 2204 causes pressurization of the actuation bladder 2204, which results in the contraction of the length of the actuation bladder 2204. The contraction of the length of the actuation bladder 2204 causes the toe of the foot to be pulled towards the leg. Thus, when the user swings the right leg forward, the toe of the foot does not drag on the ground, but is instead lifted up towards the leg of the user in a manner similar to a normal foot, also known as dorsiflexion.

The second position 2208 shows the heel strike position where the heel of the right leg 2210 makes contact with the ground and begins to take the weight of the user. From the first position 2206 to the second position 2208, the sensor bladder 2202 is deflated. As a result, the fluid from the actuation bladder 2204 flows out of the actuation bladder 2204 and back into the sensor bladder 2202. The outflow of fluid from the actuation bladder 2204 causes the actuation bladder 2204 to decompress. The decompression of the actuation bladder 2204 in turn causes the actuation bladder 2204 to increase in length from where the length was when it was compressed. Thus, the foot of the user can extend away from the leg, and make contact with the ground when the user moves forward.

The sensor bladder 2202 can be positioned at other locations instead of, or in addition to, being positioned behind the knee of the user. For example, the sensor bladder 2202 can be positioned under the foot of the leg of the user other than the one on which the actuation bladder 2204 is positioned. Specifically, the actuation bladder 2204 can be positioned on the foot of the right leg 2210, while the sensor bladder 2202 can be positioned under the foot of the left leg 2212. While FIG. 22 does not explicitly show a conduit, at least one conduit can couple the sensor bladder 2202 with the actuation bladder 2204. In some instances, the at least one conduit can run up the left leg 2212 and then down the right leg 2210 to couple the sensor bladder 2202 and the actuation bladder 2204. Referring to FIG. 22, in the first position 2206, with the sensor bladder 2202 situated below the foot of the left leg 2212 (shown in dotted line), the sensor bladder 2202 will be deflated causing the fluid from the sensor bladder 2202 to be driven into the actuation bladder 2204. The actuation bladder 2204 upon being inflated with the fluid can contract in length, pulling the right foot up while the right leg 2210 moves forward. While FIG. 22 shows the actuation bladder 2204 on only the right foot, a similar actuation bladder can be attached to the left foot as well. The sensor bladder in fluid communication with the actuation bladder on the left foot can be positioned behind the knee or positioned under the right foot.

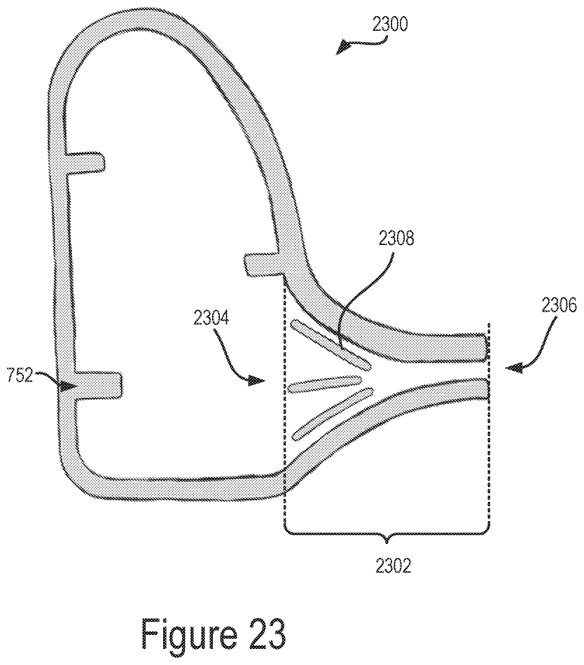

In some instances, the soft robotic systems discussed above in relation to FIGS. 1-20 can be used in combination with the soft robotic systems discussed in FIGS. 22 and 23. In contrast with traditional robotic systems that include electronic sensors and actuators to address neuropathy disorders, the soft robotic systems discussed herein are devoid of any electronic control. Instead, human power is leveraged to actuate various stimulation or actuation bladders to provide the user feedback of movement of parts of the body that are neuropathic.

In some instances, the sensor bladder, the conduit, and the stimulation bladder (or actuation bladder) can be formed as an integral unit as opposed to separable units. For example, as discussed above in relation to FIG. 11, the bladders can be formed by attaching a first membrane to a second membrane with a blocking material positioned therebetween. The blocking material can form a template of the structure to be formed. For example, the blocking material can be shaped to include the sensor bladder, the conduit, and the stimulation bladder. Thereafter, the first and the second membrane can be positioned below and above the blocking material and bonded at the perimeter of the blocking material to form integrated bladders and conduits. Thus, unlike some of the bladders discussed herein, where the bladders included a port to which the conduit was attached, the bladder and the conduit form an inseparable integrated unit. The integrated unit can be easier to install in socks, shoes, etc. The integrated unit can include at least one opening through which the fluid can be introduced into the unit. A valve can be attached to the opening to allow introduction or removal of fluid from the integrated unit. The opening can be positioned on, for example, the at least one conduit, however, the opening could be positioned anywhere based at least one ease of access and user comfort.

FIG. 23 shows another example bladder with a tubing portion. Specifically. FIG. 23 shows an example bladder 2300 that reduces forces against the bottom of the foot of the user. The example bladder 2300 can be utilized to implement the sensor bladder discussed herein. The bladder 2300 is similar to, for example, the sensor bladders discussed in relation to FIG. 7, but additionally includes a tubing portion 2302 that can represent a port that interfaces with the at least one conduit. The tubing portion 2302 has a first end 2304 and a second end 2306. The first end 2304 can be broader then the second end 2306 and can open into the interior of the bladder 2300.

The second end 2306 can be narrower than the first end 2304 and can couple with a conduit. The bladder 2300 can be positioned anywhere under the foot, but the tubing portion 2302 can be positioned such that it is wraps around the side of the foot. The broader or wider first end 2304 makes it harder to block fluid flow out of the bladder. In addition, the wider first end 2304 can be positioned to be potentially pressed down by the foot. The tubing portion 2302 includes one or more walls 2308, which can be similar to the walls discussed above in relation to FIG. 7. The one or more walls 2308 can extend between the first end 2304 and the second end 2306 and can help ensure that the tubing portion 2302 does not expand excessively and apply undue pressure to the outside of the foot. In some instances, the one or more walls 2308 may be absent.

Aspects: the following provides details of various aspects in relation to the disclosure.

Aspect 1: an apparatus, comprising: at least one sensor bladder for positioning in relation to the foot of a user; at least one stimulation bladder corresponding to the respective at least one sensor bladder, the at least one stimulation bladder positioned on the body of the user remotely from the foot; and at least one conduit fluidly coupling the respective at least one sensor bladder with the respective at least one stimulation bladder, wherein the at least one sensor bladder is configured to compress responsive to movement of the foot of the user and drive fluid from the at least one sensor bladder to the respective at least one stimulation bladder via the respective at least one conduit.

Aspect 2: the apparatus of Aspect 1, wherein the at least one sensor bladder includes a forefoot sensor bladder for positioning under the forefoot of the user and a hindfoot sensor bladder for positioning under the hindfoot of the user.

Aspect 3: the apparatus of Aspect 1, wherein the at least one sensor bladder includes a first forefoot sensor bladder for positioning under a first portion of the forefoot of the user, a second forefoot sensor bladder for positioning under a second portion of the forefoot, and a hindfoot sensor bladder for positioning under the hindfoot of the user.

Aspect 4: the apparatus of Aspect 3, wherein the first portion of the forefoot includes the bottom surface of the toes of the foot and the second portion of the forefoot includes a bottom surface of the foot between the toes and the heel, and the hindfoot of the user includes the heel of the foot.

Aspect 5: the apparatus of Aspect 3, wherein the first portion of the forefoot includes a lateral forefoot portion and the second portion of the forefoot includes a medial forefoot portion.

Aspect 6: the apparatus of Aspect 1, further comprising: a shoe insert having a foot facing surface, the at least one sensor bladder attached to the foot facing surface, the shoe insert including: a side surface defining at least one conduit port for coupling with the respective one of the at least one conduit.

Aspect 7: the apparatus of Aspect 1, further comprising: a shoe insert having a foot facing surface and an opposite shoe facing surface, the at least one sensor bladder attached to the shoe facing surface, the shoe insert including: a side surface defining at least one conduit port for coupling with the respective one of the at least one conduit.

Aspect 8: the apparatus of any one of Aspects 6 and 7, further comprising: a shoe for receiving the shoe insert, the shoe including a sole having an outer sidewall that defines at least one opening to allow the at least one conduit to pass through the sole and couple with the at least one conduit port.

Aspect 9: the apparatus of Aspect 1, further comprising: a lower extremity garment having an inner surface and an outer surface, wherein the at least one sensor bladder is attached to the outer surface or the inner surface of the lower extremity garment.

Aspect 10: the apparatus of Aspect 9, wherein the lower extremity garment comprises a sock, a compression sleeve, or a brace; and/or the lower extremity garment is machine-washable or waterproof.

Aspect 11: the apparatus of any one of Aspects 9 and 10, wherein the lower extremity garment includes an upper portion that extends beyond the ankle of the foot and wherein the at least one stimulation bladder is attached to the upper portion of the lower extremity garment.

Aspect 12: the apparatus of Aspect 1, wherein the at least one sensor bladder includes a first membrane and a second membrane and wherein portions of the inner surfaces of the first membrane and the second membrane are attached to each other.

Aspect 13: the apparatus of Aspect 1, further comprising one or more walls that extend between a first inner surface and an opposing second inner surface of the at least one sensor bladder, and wherein each bladder of the at least one sensor bladder has a contiguous volume.

Aspect 14: the apparatus of Aspect 13, wherein the at least one stimulation bladder comprises one or more walls that extend inwardly from a perimeter of the at least one stimulation bladder.

Aspect 15: the apparatus of Aspect 1, wherein the at least one sensor bladder includes an expander positioned between two inner surfaces, wherein the expander is configured to compress responsive to compressive force from the foot of the user and to expand and draw fluid back into the at least one sensor bladder responsive to reduction in compressive force from the foot.

Aspect 16: the apparatus of Aspect 1, further comprising a wearable leg-wrap with elastic lining, wherein the at least one stimulation bladder is positioned between the wearable leg-wrap and the elastic lining.

Aspect 17: the apparatus of Aspect 1, wherein the at least one stimulation bladder incudes a ring-shaped bladder wrapped circumferentially around the leg of the user.

Aspect 18: the apparatus of Aspect 1, wherein the at least one stimulation bladder includes two or more ring-shaped bladders wrapped circumferentially around the leg of a user.

Aspect 19: the apparatus of any one of Aspects 17 and 18, wherein the at least one stimulation bladder includes McKibben air muscles.

Aspect 20: the apparatus of any one of Aspects 17 and 18, wherein the at least one stimulation bladder includes pneumatic artificial muscles.

Aspect 21: the apparatus of Aspect 1, wherein the at least one stimulation bladder includes two or more stimulation bladders, each having a width that is less than a half the circumference of a portion of the leg where the respective one of the two or more stimulation bladders is disposed.

Aspect 22: the apparatus of Aspect 1, wherein the at least one stimulation bladder includes a plurality of stimulation bladders and wherein the plurality of stimulation bladders are positioned on the leg of the user at approximately the same distance from the base of the foot.

Aspect 23: the apparatus of Aspect 1, wherein the at least one stimulation bladder includes a plurality of stimulation bladders and wherein the plurality of stimulation bladders are positioned on the leg of the user at different distances from the base of the foot.

Aspect 24: the apparatus of Aspect 1, wherein the at least one stimulation bladder includes a plurality of stimulation bladders and wherein at least two of the plurality of stimulation bladders are positioned on opposite sides of the leg of the user.

Aspect 25: the apparatus of Aspect 1, wherein the at least one stimulation bladder includes a wall positioned in front of a fluid port of the at least one stimulation bladder.

Aspect 26: the apparatus of Aspect 1, wherein the fluid includes air.

Aspect 27: the apparatus of Aspect 1, wherein the fluid incudes a liquid.

Aspect 28: the apparatus of Aspect 1, further comprising at least one valve coupled with the respective one of at least one conduit to allow for fluid to be removed or entered.

Aspect 29: the apparatus of Aspect 1, wherein the at least one sensor bladder is formed of material including thermoplastic polyurethane (TPU) film or thermoset plastics.

Aspect 30: the apparatus of Aspect 1, wherein the at least one conduit is formed of material including perfluoroalkoxy alkanes (PFA) or thermoset plastics.

Aspect 31: the apparatus of Aspect 1, wherein the at least one sensor bladder, the at least one stimulation bladder, and the at least one conduit form an integrated unit, the apparatus further comprising a valve attached to an opening into the integrated unit.

Aspect 32: the apparatus of Aspect 1, wherein the at least one sensor bladder includes a tubing portion having a first end and a second end, the first end being broader than the second end, the first end opening into the interior of the at least one sensor bladder and the second end coupling with the at least one conduit.

Aspect 33: the apparatus of Aspect 32, further comprising at least one wall positioned within the tubing portion extending between the first end and the second end.

Aspect 34: the apparatus of Aspect 1, wherein the at least one stimulation bladder is positioned anywhere at the body of the user.

Aspect 35: the apparatus of Aspect 1, wherein the at least one sensor bladder and the at least one stimulation bladder are shaped differently.

Aspect 36: the apparatus of Aspect 1, wherein each of the one or more walls form a hollow slot.

Aspect 37: the apparatus of Aspect 36, wherein the hollow slot is substantially rectangular in shape.

Aspect 38: The apparatus of Aspect 1, further comprising a wearable leg-wrap with elastic lining, wherein the at least one stimulation bladder or the at least one sensor bladder is sandwiched by the wearable leg-wrap against a leg of the user.

Aspect 39: an apparatus, comprising: a first sensor bladder positioned behind a knee of a user, the first sensor bladder configured to compress when the user is about to take a step forward; a actuation bladder extending between the ankle and the top of a foot of the user, the actuation bladder configured to contract in length in response to pressurization, and a conduit extending between the first sensor bladder and the actuation bladder.

Aspect 40: the apparatus of Aspect 39, wherein the first sensor bladder is positioned behind the knee of a first leg of the user, and wherein the actuation bladder is positioned on the foot of a second leg of the user.

Aspect 41: the apparatus of Aspect 39, wherein the first sensor bladder is positioned under the foot of a first leg of the user, and wherein the actuation bladder is positioned on the foot of a second leg of the user.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

1. An apparatus, comprising:
at least one sensor bladder for positioning in relation to the foot of a user;
at least one stimulation bladder corresponding to the respective at least one sensor bladder, the at least one stimulation bladder positioned on the body of the user remotely from the foot; and
at least one conduit fluidly coupling the respective at least one sensor bladder with the respective at least one stimulation bladder,
wherein the at least one sensor bladder is configured to compress responsive to movement of the foot of the user and drive fluid from the at least one sensor bladder to the respective at least one stimulation bladder via the respective at least one conduit.

2. The apparatus of claim 1, wherein the at least one sensor bladder includes a forefoot sensor bladder for positioning under the forefoot of the user and a hindfoot sensor bladder for positioning under the hindfoot of the user.

3. The apparatus of claim 1, wherein the at least one sensor bladder includes a first forefoot sensor bladder for positioning under a first portion of the forefoot of the user, a second forefoot sensor bladder for positioning under a second portion of the forefoot, and a hindfoot sensor bladder for positioning under the hindfoot of the user.

4. The apparatus of claim 3, wherein the first portion of the forefoot includes the bottom surface of the toes of the foot and the second portion of the forefoot includes a bottom surface of the foot between the toes and the heel, and the hindfoot of the user includes the heel of the foot.

5. The apparatus of claim 3, wherein the first portion of the forefoot includes a lateral forefoot portion and the second portion of the forefoot includes a medial forefoot portion.

6. The apparatus of claim 1, further comprising:
a shoe insert having a foot facing surface, the at least one sensor bladder attached to the foot facing surface, the shoe insert including:
a side surface defining at least one conduit port for coupling with the respective one of the at least one conduit.

7. The apparatus of claim 1, further comprising:
a shoe insert having a foot facing surface and an opposite shoe facing surface, the at least one sensor bladder attached to the shoe facing surface, the shoe insert including:
a side surface defining at least one conduit port for coupling with the respective one of the at least one conduit.

8. The apparatus of claim 6, further comprising:
a shoe for receiving the shoe insert, the shoe including a sole having an outer sidewall that defines at least one opening to allow the at least one conduit to pass through the sole and couple with the at least one conduit port.

9. The apparatus of claim 1, further comprising:
a lower extremity garment having an inner surface and an outer surface, wherein the at least one sensor bladder is attached to the outer surface or the inner surface of the lower extremity garment.

10. The apparatus of claim 9, wherein:
the lower extremity garment comprises a sock, a compression sleeve, or a brace; and/or
the lower extremity garment is machine-washable or waterproof.

11. The apparatus of claim 9, wherein the lower extremity garment includes an upper portion that extends beyond the ankle of the foot and wherein the at least one stimulation bladder is attached to the upper portion of the lower extremity garment.

12. The apparatus of claim 1, wherein the at least one sensor bladder includes a first membrane and a second membrane and wherein portions of the inner surfaces of the first membrane and the second membrane are attached to each other.

13. The apparatus of claim 1, further comprising one or more walls that extend between a first inner surface and an opposing second inner surface of the at least one sensor bladder, and wherein each bladder of the at least one sensor bladder has a contiguous volume.

14. The apparatus of claim 13, wherein the at least one stimulation bladder comprises one or more walls that extend inwardly from a perimeter of the at least one stimulation bladder.

15. The apparatus of claim 1, wherein the at least one sensor bladder includes an expander positioned between two inner surfaces, wherein the expander is configured to compress responsive to compressive force from the foot of the user and to expand and draw fluid back into the at least one sensor bladder responsive to reduction in compressive force from the foot.

16. The apparatus of claim 1, further comprising a wearable leg-wrap with elastic lining, wherein the at least one stimulation bladder is positioned between the wearable leg-wrap and the elastic lining to support fluid flow of the fluid back to the sensory bladder.

17. The apparatus of claim 1, wherein the at least one stimulation bladder includes a ring-shaped bladder wrapped circumferentially around the leg of the user.

18. The apparatus of claim 1, wherein the at least one stimulation bladder includes two or more ring-shaped bladders wrapped circumferentially around the leg of a user.

19. The apparatus of claim 17, wherein the at least one stimulation bladder includes McKibben air muscles.

20. The apparatus of claim 17, wherein the at least one stimulation bladder includes pneumatic artificial muscles.

21-40. (canceled)
